SEQUENCE LISTING (36 sequences)

SEQ ID NO: 1 - DNA, 21 bp, Artificial
  PRIMER
gccatcctggctgtcgagaag

SEQ ID NO: 2 - DNA, 21 bp, Artificial
  PRIMER
gagccggatggactcgaagcg

SEQ ID NO: 3 - DNA, 21 bp, Artificial
  PRIMER
acaatgcacatcgaaaggaga

SEQ ID NO: 4 - DNA, 21 bp, Artificial
  PRIMER
tcctggtatttctacagtcct

SEQ ID NO: 5 - DNA, 21 bp, Artificial
  PRIMER
atagagaaagaagaatgtcgt

SEQ ID NO: 6 - DNA, 21 bp, Artificial
  PRIMER
gtgagcacagccgggcactct

SEQ ID NO: 7 - DNA, 21 bp, Artificial
  PRIMER
agcttggccatatctgatatg

SEQ ID NO: 8 - DNA, 21 bp, Artificial
  PRIMER
gatgtagcggtccgcagcaat

SEQ ID NO: 9 - DNA, 27 bp, Artificial
  PRIMER
atgacacctatcaggagtttgaagaag

SEQ ID NO: 10 - DNA, 27 bp, Artificial
  PRIMER
gatgcggagcagctctaggttagattt

SEQ ID NO: 11 - DNA, 22 bp, Artificial
  PRIMER
gggttcattaccaaggccatca

SEQ ID NO: 12 - DNA, 21 bp, Artificial
  PRIMER
ttcaggatgaacctggctgac

SEQ ID NO: 13 - DNA, 21 bp, Artificial
  PRIMER
gagtcagcctttgggaggaac

SEQ ID NO: 14 - DNA, 21 bp, Artificial
  PRIMER
tggtggtggtggtgctgcgta

SEQ ID NO: 15 - DNA, 20 bp, Artificial
  PRIMER
acagctgctgatttcaagca

SEQ ID NO: 16 - DNA, 20 bp, Artificial
  PRIMER
acaaagctcctacttgctca

SEQ ID NO: 17 - DNA, 21 bp, Artificial
  PRIMER
ccctaagcagcgcagcaagac

SEQ ID NO: 18 - DNA, 21 bp, Artificial
  PRIMER
gatgagtggtcggttctggcc

SEQ ID NO: 19 - DNA, 21 bp, Artificial
  PRIMER
tccgctcctgatgtgcaggat

SEQ ID NO: 20 - DNA, 21 bp, Artificial
  PRIMER
ggaccttagtggagtgggata

SEQ ID NO: 21 - DNA, 18 bp, Artificial
  PRIMER
gaaggtgaaggtcggagt

SEQ ID NO: 22 - DNA, 20 bp, Artificial
  PRIMER
gaagatggtgatgggatttc

SEQ ID NO: 23 - DNA, 20 bp, Artificial
  PRIMER
cgtcttcaccaccatggaga

SEQ ID NO: 24 - DNA, 20 bp, Artificial
  PRIMER
cggccatcacgccacagttt

SEQ ID NO: 25 - DNA, 20 bp, Artificial
  PRIMER
agggaggaaagcagactgac

SEQ ID NO: 26 - DNA, 20 bp, Artificial
  PRIMER
ccagtcatttccacaccact

SEQ ID NO: 27 - DNA, 24 bp, Artificial
  PRIMER
tgtgatgtgggaaagcaggaggat

SEQ ID NO: 28 - DNA, 24 bp, Artificial
  PRIMER
acatgtgctggtctctggttacga

SEQ ID NO: 29 - DNA, 20 bp, Artificial
  PRIMER
cctgaagcagaagaggatca

SEQ ID NO: 30 - DNA, 20 bp, Artificial
  PRIMER
ccgcagcttacacatgttct

SEQ ID NO: 31 - DNA, 20 bp, Artificial
  PRIMER
aaccagcaaggaaaatccaa

SEQ ID NO: 32 - DNA, 20 bp, Artificial
  PRIMER
aggagcaggacgaagatgac

SEQ ID NO: 33 - DNA, 20 bp, Artificial
  PRIMER
accagctcgcagacctacat

SEQ ID NO: 34 - DNA, 20 bp, Artificial
  PRIMER
gtgggaggaagaggtaacca

SEQ ID NO: 35 - DNA, 26 bp, Artificial
  PRIMER
ggagaccagcaagtattgtccttttg

SEQ ID NO: 36 - DNA, 22 bp, Artificial
  PRIMER
cattgctgctgggcatcgtaag

(19) United States
(12) Patent Application Publication
(10) Pub. No.: US 2010/0062477 A1
(43) Pub. Date: Mar. 11, 2010

(54) METHODS OF ISOLATING AND PROPAGATING STEM CELLS FROM BENIGN TUMORS

(75) Inventor: John S. Yu, Los Angeles, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/514,778

(22) PCT Filed: Nov. 28, 2007
(86) PCT No.: PCT/US07/85799

§ 371 Date: May 13, 2009

Related U.S. Application Data
(60) Provisional application No. 60/867,472, filed on Nov. 28, 2006.

(51) Int. Cl. C12Q 1/02; C12N 5/00; C12N 5/02; C12P 21/06

(52) U.S. Cl. 435/29; 435/325; 435/377; 435/70.3

(57) ABSTRACT

The present invention describes benign tumor stem cells, a method of isolating the benign tumor stem cells, a method of generating the benign tumor stem cells and a method of using the benign tumor stem cells. Uses of the benign tumor stem cells, such as pituitary stem cells include but are not limited to producing pituitary hormones and identifying drugs to treat pituitary disease conditions or pituitary-related disease conditions.

### FIELD OF INVENTION

This invention relates to the isolation and propagation of stem cells from benign tumors, and to the use of these cells.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Research involving stem cells within benign tumors is of interest in the art. For example, precancerous stem cell clones were established from the spleen of a mouse with dendritic cell-like lymphoma. (Shen et al., The Precancerous Stem Cells Can Be a Common Progenitor for Cancer Cells and Mesenchymal Supporting Cells in Malignant Tumors, FASEB J. (2006), 20(4, Pt. 1), p. A217.) These clones were bi-potent, with benign and malignant differentiation. Shen et al. were attempting to explore the contribution of the precancerous cells to tumor components in mice with lymphoma. Also of note, increased activity of aldehyde dehydrogenase has been correlated with identification of benign stem cells in samples from acute myeloid leukemia. (Pearce et al., Characterization of Cells with a High Aldehyde Dehydrogenase Activity from Cord Blood and Acute Myeloid Leukemia Samples, Stem Cells (2005), 23(6), pp. 752-760.)

Further of note, the role of breast cancer resistance protein in protecting putative benign prostate stem cells and malignant prostate tumor stem cells against androgen deprivation and in affecting recurrence of prostate cancer have been explored. (Huss et al., Breast Cancer Resistance Protein-mediated Efflux of Androgen in Putative Benign and Malignant Prostate Stem Cells, Canc. Res. (2005), 65(15), pp. 6640-6650.) Last, research suggests that a cell line derived from a benign ovarian, three-germ layer immature teratoma had progenitor or stem cells that could differentiate into nerve or glial cells. (Yokose et al., Establishment and Characterization of a Nerve Cell Line (NC-HIMT) from HIMT Cells Derived from a Human Ovarian Immature Teratoma with Special Reference to the Induction of Neuron Differentiation by Retinoic Acid, Hum. Cell (2004), 17(1), pp. 59-66.)

However, none of the above research related to a method of isolating stem cells from benign tumors or a method of creating a benign tumor stem cell line. With the interest in these cells and the potential value of research products, there exists a need in the art for a method of isolating and propagating benign tumor stem cells.

### SUMMARY OF THE INVENTION

The following embodiments and aspects thereof are described and illustrated in conjunction with compositions and methods which are meant to be exemplary and illustrative, not limiting in scope.

The present invention provides for an isolated benign tumor stem cell. In one embodiment, the isolated benign tumor stem cell may be a pituitary tumor stem cell. In a particular embodiment, the pituitary tumor stem cell may be a pituitary adenoma stem cell. The pituitary adenoma stem cell may be from a pituitary adenoma is selected from the group consisting of prolactinoma, somatotropic adenoma, adrenocorticotropic hormone (“ACTH”)-secreting adenoma, gonadotrophic adenoma, thyrotropic adenoma, null cell adenoma, and combinations thereof. In another embodiment, the isolated benign tumor stem cell may be a meningioma stem cell. In various embodiments, the isolated benign tumor stem cell may be obtained by: providing benign tumor tissue; washing the benign tumor tissue; dissecting the benign tumor tissue; digesting the benign tumor tissue; triturating the benign tumor tissue to dissociate benign tumor cells; culturing the benign tumor cells in a medium comprising EGF and bFGF whereby the benign tumor cells growing as spheres are identified as benign tumor stem cells; and selecting the benign tumor stem cell.

The present invention also provides for a method of obtaining a population of benign tumor cells, comprising: providing a population of benign tumor stem cells; and culturing the population of benign tumor stem cells in differentiation culture medium wherein the population of benign tumor stem cells are induced to differentiate into benign tumor cells. The differentiation culture medium may comprise DMEM/F12, glutamine, horse serum, and fetal bovine serum. In various embodiments, the benign tumor cells of this method may be pituitary adenoma cells or meningioma cells. In particular embodiments, the pituitary adenoma cells may be selected from the group consisting of prolactinoma cells, somatotropic adenoma cells, adrenocorticotropic hormone (“ACTH”)-secreting adenoma cells, gonadotrophic adenoma cells, thyrotropic adenoma cells, null cell adenoma cells, and combinations thereof.

The present invention also provides for a method of producing a pituitary hormone, comprising: providing a population of pituitary stem cells and/or pituitary cells obtained by differentiation of pituitary stem cells; culturing the population in a culture medium; and isolating the pituitary hormone from the culture medium or the intracellular contents of the pituitary stem cells and/or pituitary cells obtained by differentiation of pituitary stem cells. The pituitary hormone produced may be prolactin, growth hormone, adrenocorticotropic hormone, sexual hormone, or combinations thereof. The population of cells may be prolactinoma cells, somatotropic adenoma cells, adrenocorticotropic hormone (“ACTH”)-secreting adenoma cells, gonadotrophic adenoma cells, thyrotropic adenoma cells, null cell adenoma cells, or combinations thereof.

The present invention also provides for a method of identifying a drug to treat a benign tumor disease condition or a benign tumor related disease condition, comprising: providing a population of benign tumor stem cells and/or benign tumor cells obtained by differentiation of benign tumor stem cells; culturing the population in a culture medium; adding a test compound to the culture medium; and determining the effect of the test compound on the population, wherein a test compound having a desired effect is identified as a drug capable of treating the benign tumor disease condition or the benign tumor-related disease condition. In various embodiments, benign tumor disease condition or the benign tumor-related disease condition may be a pituitary disease condition, a pituitary-related disease condition, a meningioma, or a meningeal related disease condition. In various embodiments, the pituitary disease condition or the pituitary-related disease condition may be pituitary adenoma, amenorrhea, galactorrhea, infertility, hypogonadism, gigantism, acromegaly, Cushing's disease, hyperthyroidism or combinations thereof. In a particular embodiment, the pituitary stem cells may be obtained from a pituitary adenoma. In various embodiments, the pituitary adenoma cells may be prolactinoma cells, somatotropic adenoma cells, adrenocorticotropic hormone (“ACTH”)-secreting adenoma cells, gonadotrophic adenoma cells, thyrotropic adenoma cells, null cell adenoma cells, or combinations thereof. In various embodiments, the population of benign tumor stem cells may be obtained by: providing benign tumor tissue; washing the benign tumor tissue; dissecting the benign tumor tissue; digesting the benign tumor tissue; triturating the benign tumor tissue to dissociate benign tumor cells; culturing the benign tumor cells in a medium comprising EGF and bFGF whereby the benign tumor cells growing as spheres are identified as benign tumor stem cells; and selecting the benign tumor stem cell.

The present invention also provides for a kit for producing a pituitary hormone using pituitary stem cells, comprising: a population of pituitary stem cells and/or pituitary cells obtained by differentiation of pituitary stem cells; instructions to use the population to produce the pituitary hormone comprising: instructions to culture the population in a culture medium; and instructions to isolate the pituitary hormone from the culture medium or the intracellular contents of the population. In various embodiments, the pituitary hormone produced may be prolactin, growth hormone, adrenocorticotropic hormone, sexual hormone, or combinations thereof. In various embodiments, the population may be a population of cells selected from prolactinoma cells, somatotrophic adenoma cells, adrenocorticotropic hormone (“ACTH”)-secreting adenoma cells, gonadotrophic adenoma cells, thyrotropic adenoma cells, null cell adenoma cells or combinations thereof.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts pituitary adenoma cells in accordance with an embodiment of the present invention. (A) Pituitary adenoma stem cells. (B) After withdrawal of growth factors, cells start to differentiate. (C) Daughter cells growing adherent in presence of 10% fetal bovine serum.

FIG. 2 depicts immunocytochemistry of pituitary adenoma cells in accordance with an embodiment of the present invention. Differentiated pituitary adenoma stem cells were subjected to immunostaining with the following antibodies: (A) Nestin; (C) β-III tubulin; (E) GFAP; and (G) S100. B, D, F and H depict the corresponding DAPI staining.

FIG. 3 depicts prolactin concentration in pituitary adenoma stem cells in accordance with an embodiment of the present invention. Tumor stem cells and daughter adherent cells from pituitary adenomas were plated at the density of 5×10⁵ cells/ml. The conditioned medium was collected at different time points as indicated in the graph and subjected to ELISA immunoassay. The results are the average ± standard deviation of two independent experiments and all values are calculated by subtracting the blank and the value of un-conditioned medium.

FIG. 4 depicts growth hormone concentration in pituitary adenoma stem cells in accordance with an embodiment of the present invention. Tumor stem cells and daughter adherent cells from pituitary adenomas were plated at the density of 5×10⁵ cells/ml. The conditioned medium was collected at different time points as indicated in the graph and subjected to ELISA immunoassay. The results are the average ± standard deviation of two independent experiments and all values are calculated by subtracting the blank and the value of un-conditioned medium.

FIG. 5 depicts semiquantitative RT-PCR in pituitary adenoma stem and daughter adherent cells in accordance with an embodiment of the present invention. RNA extraction and retro-transcription were performed as described herein. As controls, RNA from normal human liver (negative control) and normal human pituitary (positive control) were used. A: GAPDH, 226 bp; B: PRL, 276 bp; C: Pit-1, 304 bp; D: GH, 161 bp; E: GATA-2, 163 bp. Lane 1: Marker VI (Roche); Lane 2: Normal human liver; Lane 3: Normal human pituitary; Lane 4: Pituitary adenoma No. 1 Adherent cells; Lane 5: Pituitary adenoma No. 1 Stem cells; Lane 6: Pituitary adenoma No. 2 Adherent cells; Lane 7: Pituitary adenoma No. 2 Stem cells; Lane 8: Pituitary adenoma No. 3 Adherent cells; Lane 9: Pituitary adenoma No. 3 Stem cells; Lane 10: Blank. Primer sequences and annealing temperatures are reported in Table 2.

FIG. 6 depicts growth hormone concentration in pituitary adenoma stem cell derived tumor in mouse brain in accordance with an embodiment of the present invention. Six weeks after the injection, one mouse from each group was sacrificed and the brain rapidly removed. Under a dissection microscope, the injection area was cut and homogenized in sterile PBS 1×. The ELISA immunoassay was performed in the supernatant fraction of the homogenate. As a control, a tumor from U87 injected mice was used and processed as described herein.

FIG. 7 depicts immunohistochemistry of mouse brain sections in accordance with an embodiment of the present invention. (A) Hematoxylin & eosin staining of a brain section from a mouse injected with tumor stem cells derived from a somatotroph pituitary adenoma. (B) Staining with growth hormone antibody showed positive labeling of injected cells.

FIG. 8 depicts primary pituitary adenoma cells cultured in defined neural stem cell medium with growth factors in accordance with various embodiments of the present invention. Sphere-growing cells can be observed in the primary cells after 7-14 days culture. In the cultures, some areas were growing monolayer cells (a & b). However, sphere-forming cells were also observed in the cultures (c & d). These spheres were morphologically similar to cancer stem cell spheres in human glioblastoma cultures. The sphere-growing cells in the culture became free-floating spheres as the culture continued (e & f). The free-floating spheres were passaged in defined neural stem cell culture media for more than 30 passages without morphological and cell doubling-time changes. The free-floating spheres formed sub-spheres after dissociating into single cells (g & h). The figures shown on the left panel were from pituitary adenoma No. 2, which was a null-cell macroadenoma (a, c, e & g). The figures shown on the right panel were from pituitary adenoma No. 3, which was a somatotroph GH-positive adenoma (b, d, f & h).

FIG. 9 depicts the self-renewal ability of the tumor spheres analyzed by sub-sphere assay in accordance with various embodiments of the present invention. The sub-sphere forming efficiency was quantified in different passages of the tumor spheres.

FIG. 10 depicts sub-spheres formed from single mother cell (from pituitary adenoma No. 2) of tumor spheres expressing stem cell markers and producing hormones in accordance with various embodiments of the present invention. The sub-spheres expressed stem cell marker genes. Nestin positive spheres were observed as stained in green (a & c). Also, CD133 positive spheres were demonstrated as red (d & f). DAPI were used to localize cell nuclei (b & e). The overlay images are also shown (c & f).

FIG. 11 depicts sub-spheres formed from single mother cell (from pituitary adenoma No. 3) of tumor spheres expressing stem cell markers and producing hormones in accordance with various embodiments of the present invention. The sub-spheres expressed stem cell marker genes. Nestin positive spheres were observed as stained in green (a & c). Also CD133 positive spheres were demonstrated as red (d & f). Some growth hormone positive cells were identified around the negative stained tumor spheres (h & j). DAPI were used to localize cell nuclei (b & e). The overlay images are also shown (c, f & j).

FIG. 12 depicts pituitary tumor stem cells forming spheres resembling neurospheres in accordance with various embodiments of the present invention. Tumor spheres from two pituitary tumors (A and B) are shown.

FIG. 13 depicts adherent pituitary tumor cells differentiated from pituitary tumor stem cells in accordance with various embodiments of the present invention. Tumor spheres were switched to differentiation medium and grown for 7-10 days. Two clones (A and B) are shown.

FIG. 14 depicts hormone production by pituitary tumor spheres (open bars) and differentiated cells (closed bars) stimulated with hypothalamus hormones in accordance with various embodiments of the present invention. Upon stimulation with GH-releasing factor (GHRF), PRL-releasing peptide (PRP), and Thyrotropin-Releasing Hormone (TRH) for 24 h, the secretion of GH, PRL, and TSH, respectively, by the differentiated pituitary tumor cells were determined using ELISA. ** p<0.01.

FIG. 15 depicts relative expression of pituitary-lineage transcription factors in pituitary tumor stem cells compared to that of differentiated pituitary tumor cells in accordance with various embodiments of the present invention. mRNA expression levels were determined by reverse transcription followed by quantitative PCR.

FIG. 16 depicts several stem cell related genes in accordance with various embodiments of the present invention. PTCH1, BMI1, GLi1, SOX2, NCAM and Oct4 were highly expressed on pituitary adenoma No. 3 derived clone 1 tumor stem cells and clone 2 tumor stem cells than those on their differentiated cells by real-time PCR analysis.

FIG. 17 depicts pituitary adenoma tumor spheres' ability to form new tumors in in vivo environments in accordance with various embodiments of the present invention. Tumor spheres can form new tumors upon intracranial implantation into NOD/SCID mice. The tumor-forming ability was confirmed by serial in vivo transplantations. Murine brain sections were immunofluorescence stained with human specific nuclei antibody (green) and growth hormone antibody (red). Human specific stained cells were visualized by FITC-conjugated secondary antibody (green). The growth hormone positive cells were identified by Tex-Red-conjugated secondary antibody (red). DAPI was used for identifying nuclei (blue). The overlay images are also shown.

FIG. 18 depicts the pituitary adenoma stem cell's ability to form new tumors through serial transplantation in accordance with various embodiments of the present invention. Mice brain sections were immunofluorescence stained with human specific nuclei antibody (green) and growth hormone antibody (red). Human specific positive cells were visualized by FITC-conjugated secondary antibody (green). The growth hormone positive cells were identified by Tex-Red-conjugated secondary antibody (red). DAPI was used for identifying nuclei (blue). The overlay images were shown as well.

FIG. 19 depicts tumor stem cells derived from a meningioma in accordance with an embodiment of the present invention. Little spheres are visible one week after plating (A). Daughter cells grown as adherent (B). Cells were plated as spheres and stained with nestin. Cells were also plated in differentiating conditions and stained with the indicated antibodies. Magnification is 10×. (A) meningeal tumor stem cells; (B) adherent cells; (C) nestin; (D) corresponding DAPI; (E) βIII Tubulin; (F) corresponding DAPI; (G) GFAP; (H) corresponding DAPI.

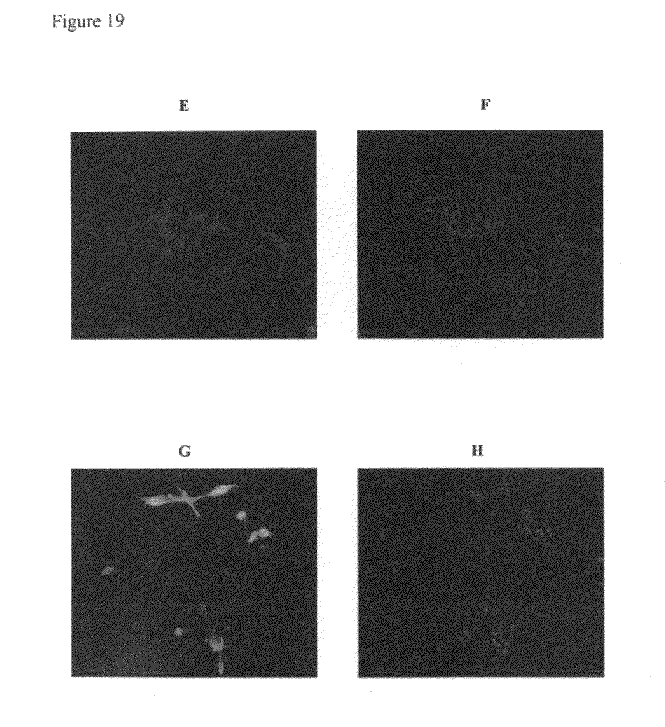

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3^{rd} ed., Cold Spring Harbor Laboratory Press (Cold Spring Harbor, N.Y. 2001), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Pituitary conditions,” “pituitary-related conditions,” “pituitary disease conditions” and “pituitary-related disease conditions” as used herein may include, but are in no way limited to, any condition or disease condition caused by or related to abnormally functioning pituitary cells, tissues or glands. Examples include, but are not limited to, benign pituitary adenomas, invasive pituitary adenomas, amenorrhea, galactorrhea, infertility, hypogonadism, gigantism, acromegaly, Cushing's disease and hyperthyroidism.

“Pituitary adenoma” as used herein refers to benign or invasive pituitary adenomas. Examples of pituitary adenomas include, but are not limited to: prolactinomas, somatotrophic adenomas, gonadotrophic adenomas, adrenocorticotropic hormone (ACTH)-secreting adenomas, thyrotropic adenomas and null cell adenomas.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Stem cell” as used herein refers to a cell that can continuously produce unaltered daughter cells and also has the ability to produce daughter cells that have different, more restricted properties.

Pituitary adenomas are common tumors of the pituitary gland and comprise 10% of intracranial neoplasms. These benign tumors which arise from the hormone producing anterior lobe cause significant morbidity from overexpression of pituitary hormones (e.g., prolactin, ACTH, TSH, and growth hormone) or from neurologic symptoms such as visual loss and headache. Despite many decades of pituitary research and extensive knowledge of cell lineage development in the embryonic pituitary, a driving pituitary stem cell which has the ability to self-renew and differentiate into the various hormone-producing cell has not been isolated from normal or adenomatous human pituitary tissue.

The inventor has, for the first time in humans, isolated stem cells from benign tumors. Furthermore, the inventor has propagated stem cell lines from meningiomas and pituitary adenomas. The etiology of benign tumors from a stem cell has not been contemplated or expected. The inventor's demonstration that isolation and propagation of benign tumor stem cells provides evidence that benign tumors have a stem cell origin. The benign tumor stem cells allow for testing of the tumor with therapeutics and for studying benign tumors to understand their genesis.

Pituitary adenomas are benign tumors and benign cells generally do not grow in culture. Thus, one of ordinary skill in the art would not have expected stem cells in benign tumors. However, the inventor believed that pituitary adenomas may contain stem cells. As such, experiments were carried out as described herein and the inventor has identified and isolated benign tumor stem cells from pituitary adenomas. The inventor has confirmed that the isolated stem cells from pituitary adenomas are indeed stem cells. With the isolation of these stem cells, the pituitary adenoma cells possess the ability to grow in culture.

Meningiomas are tumors of the brain that develop in the meninges. Meningiomas are usually benign and grow slowly. However, a small percentage of meningiomas may be malignant. The inventor also believed that benign meningiomas may contain stem cells. Thus, experiments were also conducted as described herein and the inventor has identified, isolated and propagated benign tumor stem cells from meningiomas. The inventor believes that the isolated meningioma spheres described are indeed meningioma stem cells.

Embodiments of the present invention include methods for isolating stem cells from benign tumors and methods for propagating stem cell lines from benign tumors.

The methods described herein for isolating stem cells from benign tumors may be used on any benign tumor. In one embodiment, the benign tumor is a benign brain tumor. In another embodiment, the benign tumor is a pituitary adenoma. In a particular embodiment, the pituitary adenoma is a non-invasive pituitary adenoma. In another particular embodiment, the pituitary adenoma is an invasive pituitary adenoma. Invasive pituitary adenomas are pituitary adenomas that invade into the dura mater, cranial bone, or sphenoid sinus, but are not cancerous. As used herein “benign pituitary adenoma” may refer to a non-invasive pituitary adenoma and/or an invasive pituitary adenoma. In another embodiment, the benign tumor is a meningioma. The methods described herein may be used for other benign brain tumors, including, but not limited to epidermoid, dermoid, hemangioblastoma, colloid cyst, subependymal giant cell astrocytoma, pleomorphic xanthoastrocytoma, and craniopharyngioma.

Various embodiments of the present invention provide for isolating stem cells from a benign tumor. In one embodiment, the method provides for isolating stem cells from a benign brain tumor. In a particular embodiment, the method provides for isolating stem cells from a pituitary adenoma. In another particular embodiment, the method provides for isolating stem cells from a non-invasive pituitary adenoma. In another particular embodiment, the method provides for isolating stem cells from an invasive (but non-cancerous) pituitary adenoma. In another particular embodiment, the method provides for isolating stem cells from a meningioma. In other embodiments, the method provides for isolating stem cells from benign tumors including but not limited to epidermoid, dermoid, hemangioblastoma, colloid cyst, subependymal giant cell astrocytoma, pleomorphic xanthoastrocytoma, and craniopharyngioma.

Other embodiments of the present invention provide for propagating stem cells from benign tumors. In one embodiment, the method provides for propagating stem cells from a benign brain tumor. In a particular embodiment, the method provides for propagating stem cells from a pituitary adenoma. In another particular embodiment, the method provides for propagating stem cells from a non-invasive pituitary adenoma. In another particular embodiment, the method provides for propagating stem cells from an invasive (but non-cancerous) pituitary adenoma. In another particular embodiment, the method provides for propagating stem cells from a meningioma. In other embodiments, the method provides for propagating stem cells from benign tumors including but not limited to epidermoid, dermoid, hemangioblastoma, colloid cyst, subependymal giant cell astrocytoma, pleomorphic xanthoastrocytoma, and craniopharyngioma.

Additional embodiments of the present invention include methods for using the benign stem cells or stem cell lines. One skilled in the art will readily recognize various methods for using these stem cells and stem cell lines. These benign stem cells and stem cell lines may be used for in vitro research regarding the type of tumor from which the stem cells or stem cell lines are derived. As a non-limiting example, in one embodiment, stem cells and stem cell lines isolated and propagated from pituitary adenomas may be used for studying pituitary diseases (e.g., pituitary adenomas including but not limited to prolactinomas, somatotrophic adenomas, adrenocorticotropic hormone (ACTH)-secreting adenomas, gonadrotophic adenomas, thyrotropic adenomas and null cell adenomas; invasive pituitary adenomas; amenorrhea; galactorrhea; infertility; hypogonadism; gigantism; acromegaly; Cushing's disease; and hyperthyroidism). In another embodiment, the stem cells and the stem cell lines isolated and propagated from pituitary adenomas may be used for testing therapeutic products for pituitary diseases. In another embodiment, the stem cells and the stem cell lines isolated and propagated from pituitary adenomas may be used for obtaining human pituitary hormone products (e.g., prolactin, human growth hormone, adrenocorticotropic hormone, and sexual hormones such as luteinizing hormone and follicle-stimulating hormone). All of these uses were previously impossible because no one had heretofore been able to generate a stable human stem cell line from pituitary adenoma.

In another embodiment, stem cells and stem cell lines isolated and propagated from meningiomas may be used for studying diseases of the meninges, including but not limited to meningiomas. In another embodiment, the stem cells and the stem cell lines isolated and propagated from meningiomas may be used for testing therapeutic products for diseases of the meninges, including but not limited to meningiomas.

In additional embodiments, stem cells and stem cell lines isolated and propagated from benign tumors such as epidermoid, dermoid, hemangioblastoma, colloid cyst, subependymal giant cell astrocytoma, pleomorphic xanthoastrocytoma, and craniopharyngioma may be used for studying disease or conditions caused by these tumors. In further embodiments, the stem cells and stem cell lines isolated and propagated from benign tumors such as epidermoid, dermoid, hemangioblastoma, colloid cyst, subependymal giant cell astrocytoma, pleomorphic xanthoastrocytoma, and craniopharyngioma may be used for testing therapeutic products for diseases or conditions caused by these tumors.

The methods for isolating and propagating stem cells may also be used on benign tumors originating from glands, organs, and soft tissues outside of the central nervous system. The benign stem cells and stem cell lines from non-neural benign tumors may also be used for studying diseases or disease conditions caused by the tumor.

Isolation and propagation of benign tumor stem cells and benign tumor stem cell lines may be accomplished as described by Yuan et al., “Isolation of cancer stem cells from adult glioblastoma multiforme,” Oncogene (2004) 23; 9392-9400, hereby incorporated by reference as though fully set forth in its entirety. Yuan et al. described a method of isolation of cancer stem cells. However, it was unexpected that the same process could be used for cells originating outside of the central nervous system and for benign tumor cells.

In one embodiment, benign tumor samples may be collected from surgical resection. Tissues may be washed and dissected in a dissection medium containing catalase, deferoxamine, N-Acetyl cysteine and superoxide dismutase. After digestion in trypsin for about 10 min at about 37° C., tumor tissues may be triturated. Tituration may be performed by passing the tissues in a tissue sieve and after recovering the cells by passing them through a 70 μm cell strainer. Cells may then plated at the density of about 1×10⁵ cells/ml in a medium containing DMEM/F12 (1:1) (Gibco), 10% FBS (Omega Scientific), penicillin/streptomycin (200 U/ml; Gibco) and Glutamax 1× (Gibco). After about 24-48 hour, medium may be changed with a medium containing DMEM/F12 (1:1) (Gibco), B27 1× (Gibco), penicillin/streptomycin (200 U/ml; Gibco), fungizone (250 ng/ml), EGF (20 ng/ml) and bFGF (20 ng/ml). Cells growing as spheres are selected. For each cell line, daughter cells growing adherent in 10% FBS containing medium may also be prepared.

The benign tumor stem cells and benign tumor stem cell lines of the present invention may be used to test therapeutic products and to discover and develop novel anti-tumor and anti-cancer therapies. Several types of experimental approaches can utilize these cell lines in vitro and in vivo, including conventional chemotherapies, immunomodulatory therapies, and gene therapies affecting benign tumor cells. Examples of these therapies include: cytostatic agents, biological response modifiers, cytokine expressing agents, gene therapy vector agents, immunotoxin agents, antiproliferative agents, anti-metastasis agents, and angiostatic agents.

In various embodiments, the benign tumor stem cells and benign tumor stem cell lines may be used for identifying compounds or conditions that induce or inhibit differentiation of benign tumor stem cells. The benign tumor stem cells and benign tumor stem cell lines may be useful for screening chemical agents to identify chemicals which may induce or inhibit benign tumors or other related diseases in vitro. To determine whether a chemical can induce or inhibit differentiation, the benign tumor stem cells and benign tumor stem cell lines may be cultured; for example, by plating a tissue culture plate in a medium at about 37° C. In particular embodiments, a test compound may be added to the cells with each medium change. At particular time points, the ability of the test compound to induce or inhibit differentiation of the cells may be determined.

In another embodiment, the benign tumor stem cells and benign tumor stem cell lines may be useful in methods for determining whether a compound (e.g., a chemotherapeutic agent, an anti-proliferation agent, a cytotoxic agent, etc.) or particular culture conditions can induce proliferation or inhibit proliferation of benign tumor stem cells. Particular compounds may induce or inhibit the proliferation of the benign tumor stem cells. To determine the ability of a compound to induce or inhibit proliferation in benign tumor cells, the benign tumor stem cells may be cultured. A test compound may be added to the cells with each medium change. At particular time points, the effect of the test compound on the cells may be determined.

In accordance with another embodiment of the invention, the benign tumor stem cells and benign tumor stem cell lines may be used to screen for therapeutic compounds as evidenced by a test compound's ability to modulate a biochemical activity of the cells (e.g., the cells' growth, signaling pathways, etc.).

In one exemplary application of the screening method, the cells are grown in a suitable medium and a test compound is added to the culture to determine the effect on the cells. For example, a suspension of cultured cells may be aliquoted into each of several wells, and increasing amounts of the test compound, (e.g., 0, 10, 100, 1000, 10,000 mM) are added to the wells. After a suitable incubation time, the level of the detectable marker protein in the wells is measured to determine if the compound, at any concentration, has resulted in affecting the cell.

Compounds tested may include known anti-tumor compounds. Compounds identified as anti-tumor compound candidates may be further tested in defined screening systems, such as animal model systems, to further assess the potential of the compound as an anti-tumor agent.

It will be appreciated that the screening format is readily adaptable to high-throughput screening (HTS), for example, by simultaneously screening a large number of samples in the microtiter wells of a multiwell plate, such as one having 96, 720 or larger numbers of wells. The wells may be readily assayed for a compound's effect, simply by assaying the level of the fluorescence from the cell samples at optimal fluorescence excitation and emission wavelengths. Compounds that test positive may then be retested for more precise dose response to further determine the potential value of the compound.

When a test compound modulates the level of the detectable marker and/or its activity at a pharmaceutically practical level has been identified, the compound may be further assayed to develop its pharmacological profile. Such tests may include in vitro cell-culture studies to determine the effect of the identified compound, the ability of the identified compound to inhibit proliferation, the ability of the compound to inhibit proliferation in suitable animal model systems, and the toxicology profile of the compound in animals.

In addition, when test compounds are identified, the compound may be further developed by standard drug-design or combinatorial-structure approaches to seek more active analogs, and/or compounds with reduced toxicity.

Other embodiments of the present invention utilize the benign tumor stem cells and benign tumor stem cell lines to study diseases; for example, pituitary diseases and meningeal diseases, to name a few. Example of pituitary diseases and pituitary-related diseases include but are not limited to pituitary adenomas (e.g., prolactinomas, somatotrophic adenomas, adrenocorticotropic hormone (ACTH)-secreting adenomas, gonadrotophic adenomas, thyrotropic adenomas and null cell adenomas), amenorrhea, galactorrhea, infertility, hypogonadism, gigantism, acromegaly, Cushing's disease, and hyperthyroidism. For example, the benign tumor stem cells described herein may be implanted into laboratory animals (e.g., mice, rat, etc.) for various in vivo studies. These studies may investigate the genetic or other biological etiologies of benign tumors. Other studies may test compounds or therapies for the treatment of benign tumors and related diseases.

Additional embodiments of the present invention may utilize the benign pituitary adenoma stem cells and benign pituitary adenoma stem cell lines to obtain human pituitary hormone products (e.g., prolactin, human growth hormone, adrenocorticotropic hormone, and sexual hormones such as luteinizing hormone and follicle-stimulating hormone). Cells lines as described herein may be used to produce hormone products and the hormone products may be isolated from the media or from the intracellular contents.

The present invention is also directed to kits for isolating benign tumor stem cells; kits for propagating benign tumor stem cells and stem cell lines; and kits for using the benign tumor stem cells and benign tumor stem cell lines to test therapeutic products, to study benign tumors and related diseases, and/or to obtain human pituitary hormone products.

Each kit is an assemblage of materials or components. The exact nature of the components configured in each inventive kit depends on its intended purpose. For example, some embodiments are configured for the purpose of isolating the benign tumor stem cells. Additional embodiments are configured for the purpose of propagating the benign tumor stem cells and benign tumor stem cell lines. Particular embodiments may be configured for the purpose of isolating pituitary stem cells; propagating the pituitary stem cells and stem cell lines described herein; and/or using the pituitary stem cells or stem cell lines described herein to test therapeutic products, to study pituitary diseases, pituitary-related diseases and pituitary adenomas, and/or to obtain human pituitary hormone products. Additional embodiments may be configured for the purpose of isolating meningioma stem cells; propagating the meningioma stem cells and stem cell lines described herein; and/or using the meningioma stem cells and cell lines described herein to test therapeutic products, and/or to study meningiomas and meningeal-related diseases or conditions. In some embodiments, the kits are configured particularly for mammalian subjects. In another embodiment, the kits are configured particularly for human subjects. In further embodiments, the kits are configured for veterinary animals, such as such as, but not limited to, farm animals, domestic animals, and laboratory animals.

Instructions for use may be included in the kit. “Instructions for use” typically include a tangible expression describing the technique to be employed in using the components of the kit for a desired purpose, such as for isolating the benign tumor stem cells; propagating the benign tumor stem cells and stem cell lines; and/or using benign tumor stem cells and stem cell lines to test therapeutic products and/or to study diseases. Optionally, the kits also contain other useful components, such as, buffers (e.g., PBS), growth media, tissue culture plates, multiple-well plates, flasks, chamber slides, differentiation media, stem cell media, tumor stem cell media, neural stem cell media, goat serum, fetal bovine serum, basic fibroblast growth factor, epidermal growth factor, diluents, pharmaceutically acceptable carriers, syringes, catheters, applicators, pipetting or measuring tools, or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a cryocontainer used to contain suitable quantities of benign tumor stem cells or stem cell lines described herein. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent methods or reactants without the exercise of inventive capacity and without departing from the scope of the invention.

### Example 1

### Patients

Pituitary tumor stem neurospheres were prepared from four pituitary macroadenomas null-cell, two somatotroph (growth hormone producing) pituitary adenomas and two pituitary macroadenomas with acromegaly. (see Table 1).

TABLE 1
Pituitary tumor and patients characteristics

| Tumor | Patient age | Diagnosis |
|---|---|---|
| PA 1 | 43 | microadenoma GH+ |
| PA 2 | 58 | macroadenoma null-cell |
| PA 3 | 19 | somatotroph GH+ |
| PA 4 | 40 | macroadenoma with acromegaly |
| PA 5 | 86 | macroadenoma null-cell |
| PA 6 | 64 | macroadenoma null-cell |
| PA 7 | 57 | adenoma with acromegaly |
| PA 8 | 50 | macroadenoma null-cell |

Meningioma tumor stem cells were prepared from meningiomas isolated from patients. “Meningioma 4” was isolated from a meningioma of a 42 year old female. “Meningioma 8” was isolated from a meningioma of an 85 year old female.

### Example 2

### Preparation of Tumor Stem Neurospheres from Pituitary Adenomas

Tumor stem neurospheres were prepared as described by Yuan et al. Briefly, tumor samples from eight pituitary adenoma patients were collected within half an hour from the surgical resection as approved by the Institutional Review Board at Cedars Sinai Medical Center. Tissues were washed three times in PBS 1× and dissected in a dissection medium containing catalase, deferoxamine, N-Acetyl cysteine and superoxide dismutase. After digestion in trypsin for 10 min at 37° C., tumor tissues were triturated by passing them in a tissue sieve and after recovering the cells by passing in a 70 μm cell strainer. Cells were then plated at the density of 1×10⁵ cells/ml in a medium containing DMEM/F12 (1:1) (Gibco), 10% FBS (Omega Scientific), penicillin/streptomycin (200 U/ml; Gibco) and Glutamax 1× (Gibco). After 24-48 h medium was changed with a medium containing DMEM/F12 (1:1) (Gibco), B27 1× (Gibco), penicillin/streptomycin (200 U/ml; Gibco), fungizone (250 ng/ml), EGF (20 ng/ml) and bFGF (20 ng/ml). For each cell line, daughter cells growing adherent in 10% FBS containing medium were also prepared.

### Example 3

### Preparation of Tumor Stem Neurospheres from Meningiomas

Menigioma stem neurospheres were prepared as described by Yuan et al. Briefly, tumor samples from meningioma patients were collected within half an hour from the surgical resection as approved by the Institutional Review Board at Cedars Sinai Medical Center. Tissues were washed three times in PBS 1× and dissected in a dissection medium containing catalase, deferoxamine, N-Acetyl cysteine and superoxide dismutase. After digestion in trypsin for 10 min at 37° C., tumor tissues were triturated by passing them in a tissue sieve and after recovering the cells by passing in a 70 μm cell strainer. Cells were then plated at the density of 1×10⁵ cells/ml in a medium containing DMEM/F12 (1:1) (Gibco), 10% FBS (Omega Scientific), penicillin/streptomycin (200 U/ml; Gibco) and Glutamax 1× (Gibco). After 24-48 h medium was changed with a medium containing DMEM/F12 (1:1) (Gibco), B27 1× (Gibco), penicillin/streptomycin (200 U/ml; Gibco), fungizone (250 ng/ml), EGF (20 ng/ml) and bFGF (20 ng/ml). For each cell line, daughter cells growing adherent in 10% FBS containing medium were also prepared.

### Example 4

### Immunocytochemistry Analysis of Tumor Stem Neurospheres Derived from Pituitary Tumors

Neurospheres from pituitary tumors were analyzed by immunocytochemistry after growing them in poly-lysine coated chamber slides with or without growth factors for 10 days (differentiation conditions). Cells were fixed in PBS 1×/4% paraformaldehyde, and after several washing with PBS 1×, they were permeabilized in 0.1% Triton X-100 and blocked with 10% goat serum in PBS 1×. Cells were then incubated with primary antibodies: GFAP (1:200, Dako, Denmark), β-III Tubulin (1:400, Covance, Berkeley, Calif.), S-100 (1:200, Chemicon), CNPase (1:200), nestin (1:50, Chemicon, Temecula, Calif.) and CD133 (1:200, Milteny Biotech, Auburn, Calif.). After incubation with FITC-conjugated secondary antibodies (1:300) slides were counter-stained with a mounting medium containing DAPI (Vector Laboratories) before examination by fluorescence microscopy.

### Example 5

### Determination of Pituitary Hormones Production from Stem Neurospheres Derived from Pituitary Adenomas

In order to determine and quantify the pituitary hormones production from the tumor stem cells derived from the pituitary tumors, 5×10⁵ cells from each cell line were plated in 5 wells of a six-well plate. For each cell line the daughter adherent cells were plated in parallel in other plates. Conditioned medium from each cell line was collected 2, 4, 8, 16 and 24 h after the plating and kept at ±80° C. until use. ELISA kits (GH, PRL, FSH, LH, TSH were from Anogen, ACTH from R&D Systems) were used for the quantification of the pituitary hormones and the suggested protocols were followed.

Quantification of growth hormone and prolactin was also performed in brain homogenates from mice-injected with pituitary adenoma stem cells.

### Example 6

### In Vivo Injection of Pituitary Adenoma Stem Neurospheres

Pituitary adenomas neurospheres (1×10⁴) and adherent daughter cells (1×10⁵) from one null and one growth hormone-producing tumor were stereotactically injected into the right hemisphere (coordinates from Bregma: 0.5 mm anterior, 2.2 mm right, 3.0 depth) of NOD/SCID mice (The Jackson Laboratories, Bar Harbor Me., USA; n=4 for each group). Control mice (n=3) were injected with 1×10⁴ U87 glioma cell line. The experiment was repeated once with identical conditions. Mice were sacrificed at different time points and brain sections of the tumor were examined as described below. All the experiments were performed in the accordance with the Institutional Animal Care and Use Committee guidelines from Cedars Sinai Medical Center.

### Example 7

### In Vivo Injection of Meningioma Stem Neurospheres

Meningioma neurospheres (1×10⁴) and adherent daughter cells (1×10⁵) from a meningioma are stereotactically injected into the right hemisphere (coordinates from Bregma: 0.5 mm anterior, 2.2 mm right, 3.0 depth) of NOD/SCID mice (The Jackson Laboratories, Bar Harbor Me., USA; n=4 for each group). Control mice (n=3) are injected with 1×10⁴ U87 glioma cell line. Mice are sacrificed at different time points and brain sections of the tumor are examined as described below. All the experiments are performed in the accordance with the Institutional Animal Care and Use Committee guidelines from Cedars Sinai Medical Center.

### Example 8

### Immunohistochemistry Staining of Brain Sections (Injected with Pituitary Adenoma Stem Cells)

Brains from injected mice were post fixed with 10% buffered formalin at 4° C.

After inclusion in paraffin, brains were cut, mounted on slides and stained with hematoxylin-eosin. Immunohistochemical analysis was also performed to stain for the pituitary hormones as the indicated concentrations: growth hormone (1:1000; Dako), prolactin (1:1500; Dako); follicle-stimulating hormone (FSH, 1:200; Dako), luteinizing hormone (LH, 1:2000; Dako), adrenocorticotropic hormone (ACTH, 1:400; Dako), thyroid-stimulating hormone (TSH, 1:100; Dako), alpha-subunit (1:8000: Abcam).

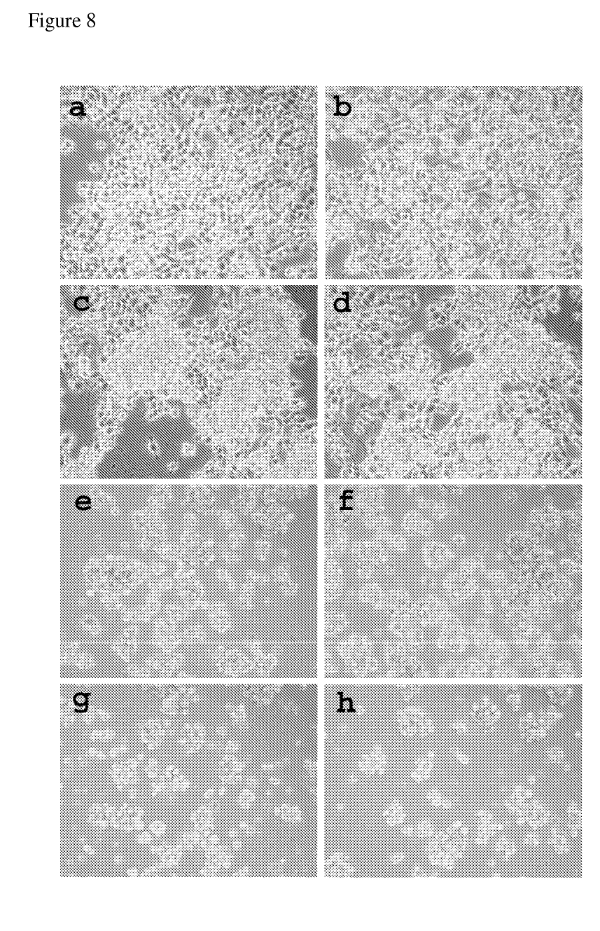

Hematoxylin & eosin staining of a brain section from a mouse injected with pituitary adenoma stem cells derived from a somatotroph pituitary adenoma was performed. (FIG. 7A.) Additionally, staining with growth hormone antibody also showed positive labeling of injected cells. (FIG. 7B.)

### Example 9

### RNA Extraction and RT-PCR Assay

RNA was extracted from pituitary adenoma stem cells and adherent cells using the Absolutely RNA miniprep kit (Statagene) following the suggested protocol. After spectrophotometer quantification two micrograms from each sample were subjected to reverse transcription using SuperScript™ II Reverse Transcriptase (Invitrogen) and Random primers (Roche). As controls, RNA from normal human liver (negative control) and normal human pituitary (positive control) were used (BioChain). The PCRs were performed in a 50 μl reaction mixture containing 2 μl cDNA as template and specific oligonucleotides listed in Table 2. Oligonucleotide sequences, annealing temperatures, primers used and sizes of the amplification products are reported in Table 2.

TABLE 2

| | | Annealing | Amplication |
|---|---|---|---|
| Primer name | Sequence | temperature | size |
| LH Fw | 5′-GCCATCCTGGCTGTCGAGAAG-3′ | 60° C. | 292 bp |
| | (SEQ ID NO. 1) | | |
| LH Rev | 5′-GAGCCGGATGGACTCGAAGCG-3′ | | |
| | (SEQ ID NO. 2) | | |
| TSH Fw | 5′-ACAATGCACATCGAAAGGAGA-3′ | 60° C. | 238 bp |
| | (SEQ ID NO. 3) | | |
| TSH Rev | 5′-TCCTGGTATTTCTACAGTCCT-3′ | | |
| | (SEQ ID NO. 4) | | |
| FSH Fw | 5′-ATAGAGAAAGAAGAATGTCGT-3′ | 55° C. | 172 bp |
| | (SEQ ID NO. 5) | | |
| FSH Rev | 5′-GTGAGCACAGCCGGGCACTCT-3′ | | |
| | (SEQ ID NO. 6) | | |
| ACTH Fw | 5′-AGCTTGGCCATATCTGATATG-3′ | 60° C. | 261 bp |
| | (SEQ ID NO. 7) | | |
| ACTH Rev | 5′-GATGTAGCGGTCCGCAGCAAT-3′ | | |
| | (SEQ ID NO. 8) | | |
| GH Fw | 5′-ATGACACCTATCAGGAGTTTGAAGAAG-3′ | 58° C. | 161 bp |
| | (SEQ ID NO. 9) | | |
| GH Rev | 5′-GATGCGGAGCAGCTCTAGGTTAGATTT-3′ | | |
| | (SEQ ID NO. 10) | | |
| PRL Fw | 5′-GGGTTCATTACCAAGGCCATCA-3′ | 58° C. | 276 bp |
| | (SEQ ID NO. 11) | | |
| PRL Rev | 5′-TTCAGGATGAACCTGGCTGAC-3′ | | |
| | (SEQ ID NO. 12) | | |
| PROP1 Fw | 5′-GAGTCAGCCTTTGGGAGGAAC-3′ | 58° C. | 237 bp |
| | (SEQ ID NO. 13) | | |
| PROP1 Rev | 5′-TGGTGGTGGTGGTGCTGCGTA-3′ | | |
| | (SEQ ID NO. 14) | | |
| Pit1 Fw | 5′-ACAGCTGCTGATTTCAAGCA-3′ | 56° C. | 304 bp |
| | (SEQ ID NO. 15) | | |
| Pit1 Rev | 5′-ACAAAGCTCCTACTTGCTCA-3′ | | |
| | (SEQ ID NO. 16) | | |
| GATA-2 Fw | 5′-CCCTAAGCAGCGCAGCAAGAC-3′ | 61° C. | 163 bp |
| | (SEQ ID NO. 17) | | |
| GATA-2 Rev | 5′-GATGAGTGGTCGGTTCTGGCC-3′ | | |
| | (SEQ ID NO. 18) | | |
| Alpha-subunit | 5′-TCCGCTCCTGATGTGCAGGAT-3′ | 58° C. | 132 bp |
| FW | (SEQ ID NO. 19) | | |
| Alpha-subunit | 5′-GGACCTTAGTGGAGTGGGATA-3′ | | |
| Rev | (SEQ ID NO. 20) | | |
| GAPDH Fw | 5′-GAAGGTGAAGGTCGGAGT-3′ | 54° C. | 226 bp |
| | (SEQ ID NO. 21) | | |
| GAPDH Rev | 5′ GAAGATGGTGATGGGATTTC-3′ | | |
| | (SEQ ID NO. 22) | | |

### Example 10

### Clonal Analysis

Cells from the pituitary adenoma were plated at a low density (1000 cells/1 ml of complete medium containing EGF/bFGF) on a 96-well plate. The presence of single spheres was checked.

### Example 11

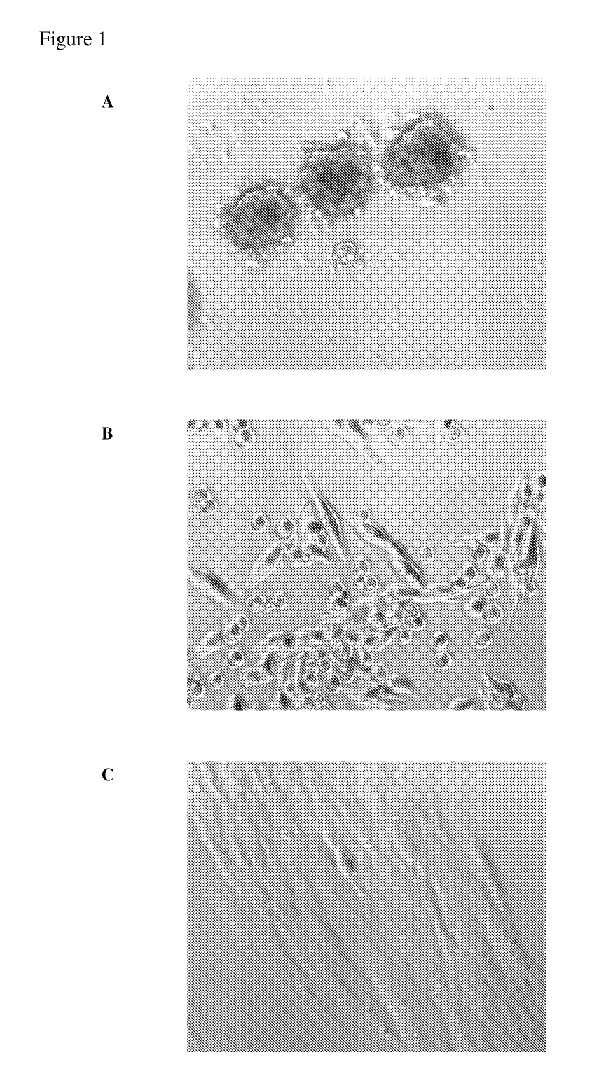

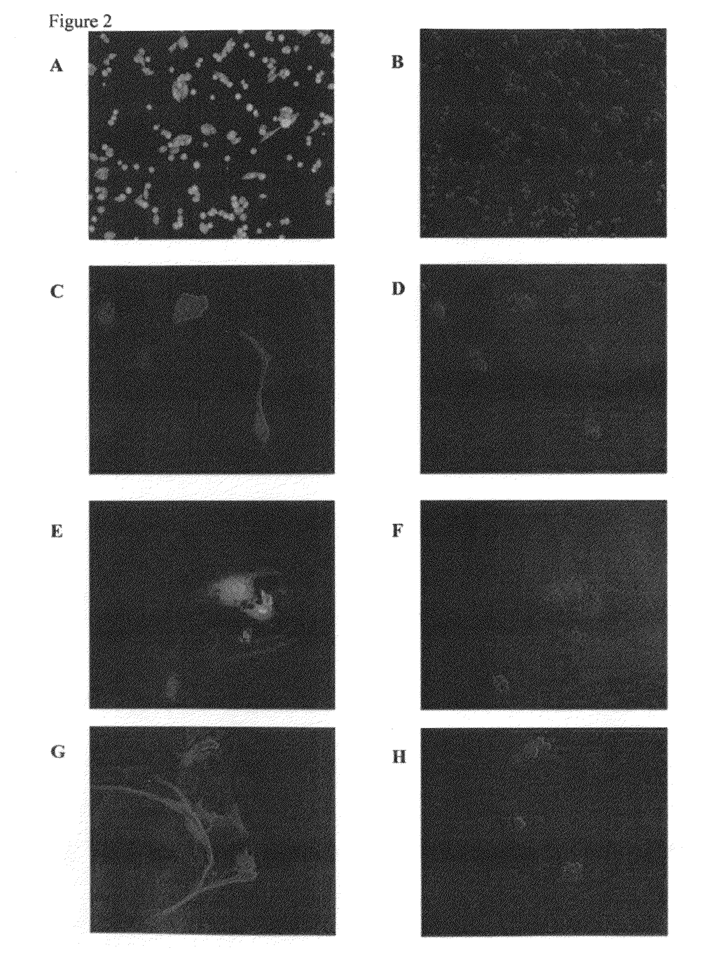

Pituitary adenomas cells growing in a medium containing EGF and bFGF showed the formation of spheres, characteristic of stem cells (FIG. 1A). The corresponding adherent daughter cells have a typical fibroblast-like phenotype (FIG. 1C). When spheres derived from these pituitary adenomas were subjected to differentiation conditions (withdrawal of growth factors and plating onto poly-lysine-coated slides, FIG. 1B), these cells showed staining for nestin (FIG. 2A), β-III tubulin (FIG. 2C) and GFAP (FIG. 2E). In addition they showed staining for S-100, a protein reported to be present in stellate cells of the pars distalis and tuberalis, in the marginal cells and in pituicytes of the neural lobe of pituitary.

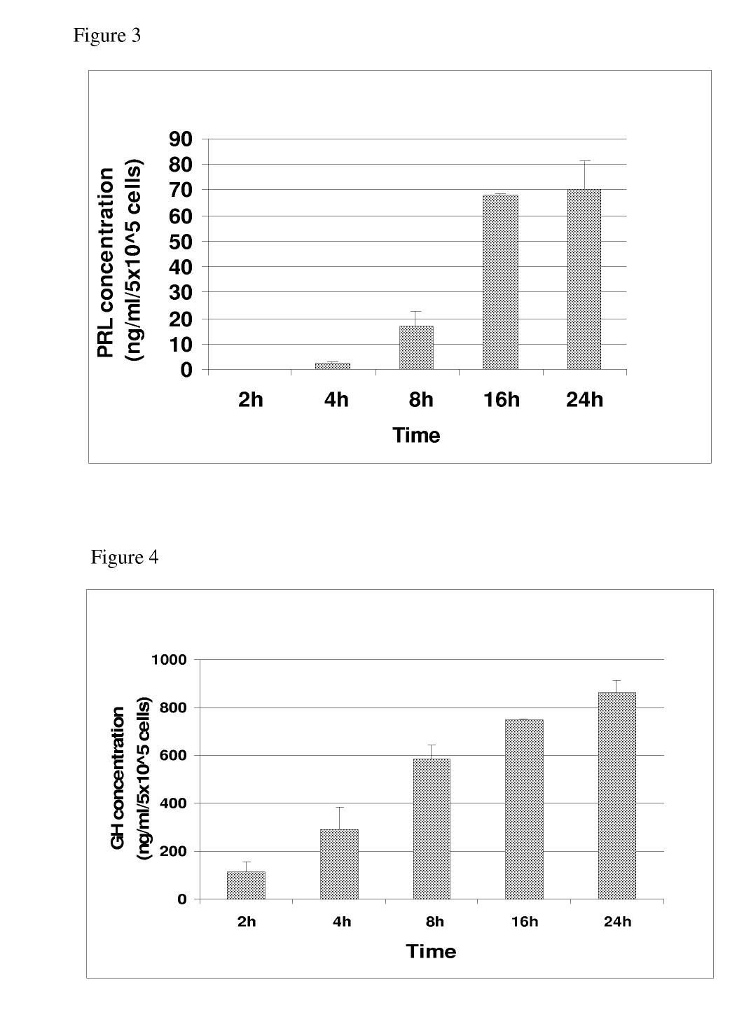

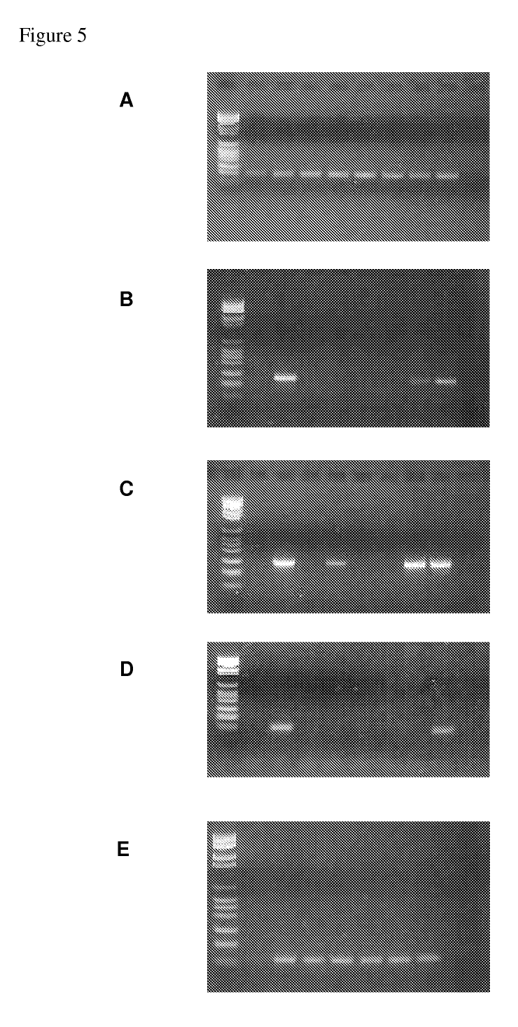

ELISA quantification of the hormone production showed a higher production of prolactin and growth hormone in one pituitary adenoma stem cell line derived from a patient with a somatotroph adenoma, while the adherent counterpart were not hormone producing (FIG. 3 and FIG. 4). The levels of the other hormones were also investigated and they remain under normal values (data not shown).

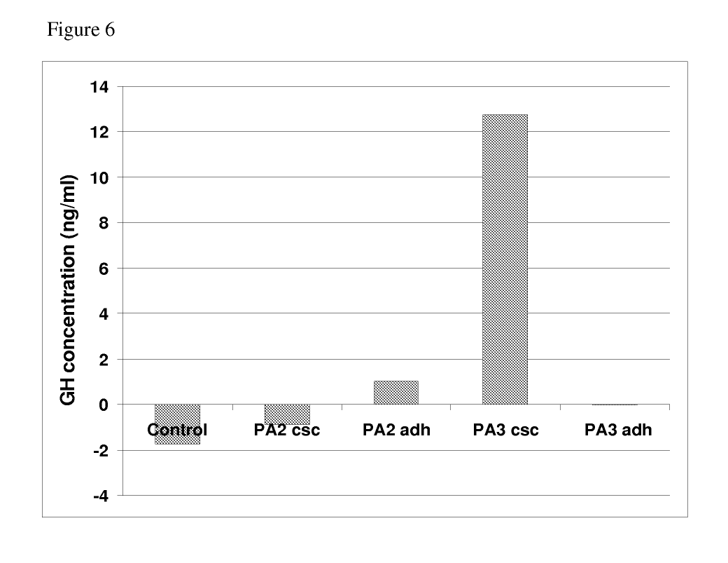

With semi-quantitative RT-PCT the inventor also detected the levels of expression of the pituitary hormones as well as that of pituitary-related transcription factors. In particular, the inventor found that the pituitary specific transcription factor Pit-1 is expressed in two of three pituitary adenoma stem cells analyzed. In one pituitary cell line, GATA-2, a zinc finger transcription factor necessary for differentiation and determination of gonadotrophs and thyrotrophs, is present only in adherent cells and not in tumor stem cells (FIG. 5).

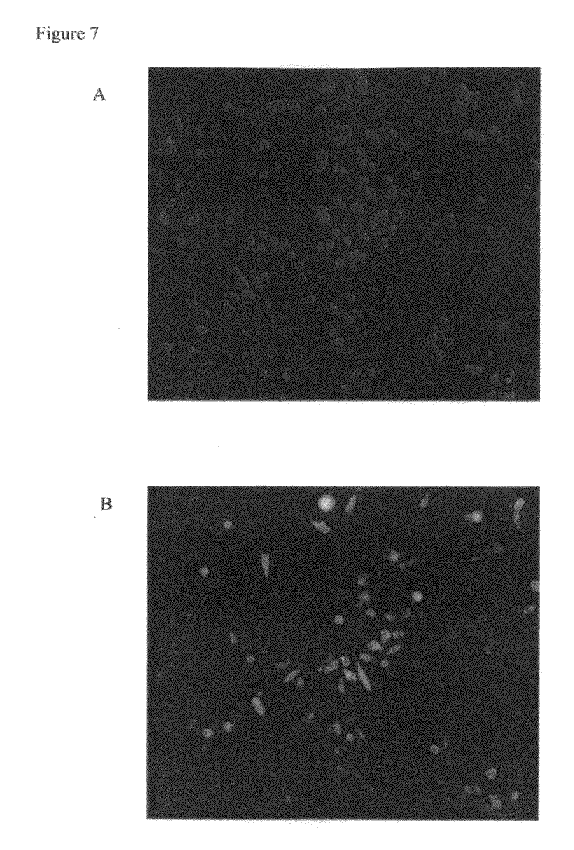

Pituitary adenomas stem derived cells were also injected in vivo in the right striatum of NOD/SCID mice. Six week after the injection part of the animals were sacrificed for histology and immunohistochemistry analysis and for the detection of growth hormone and prolactin in the mouse-tumor homogenate with ELISA immunoassay. In the brain of a mouse injected with the somatotroph adenoma-derived tumor stem cells, the inventor found the presence of the injected cells that stained positive for growth hormone. By ELISA immunoassay performed in the tumor homogenate from another mouse injected with the same cells, the inventor found a higher percentage of growth hormone with respect to control animals injected with U87 glioma cells and to mice injected with daughter adherent cells (FIG. 6). Taking into account that the ELISA assay used is specific to detect human growth hormone and do not cross react with mouse, this results confirm that these cells survive after the injection and they are able to produce the growth hormone also in vivo into the mouse host brain.

### Example 12

### Passage of Meningioma Cells

Cells growing as primary adherent tumor cells were trypsinzied, centrifuged at 1200 rpm for 5 minutes and replated onto new 75 cm² flasks with fresh medium containing 10% FBS.

Tumor stem cells were collected by centrifugation at 800 rpm for 10 minutes. The pellet was manually dissociated and the cells were replated in 25 cm² flasks with fresh medium containing EGF and bFGF.

### Example 13

### Freezing of Meningioma Cells

Adherent cells were trypsinized, collected by centrifugation at 1200 rpm for 10 minutes. The pellet was resuspended in 900 μl of complete medium containing serum and 100 μl of DMSO and transferred into a cryogenic vial and frozen at −80° C.

Tumor stem cells were collected by centrifugation at 800 rpm for 10 minutes. The pellets were resuspended in 900 μl of complete medium and 100 μl of DMSO and transferred into cryogenic vials and frozen at −80° C.

### Example 14

### DNA Extraction

DNA extraction from the whole blood of patients was performed as using QIA assay DNA mini kit available from Qiagen following the protocol provided by the kit. Four samples of 200 μl each of whole blood were process for each patient. At the end of the preparation, DNA was eluted with 100 μl of buffer AE, and a second elution with additional 100 μl of Buffer AE was performed. DNA was stored at −20° C.

### Example 15

### Fixation with 4% Paraformaldehyde of Cells

Tumor stem cells and adherent cells from meningiomas were fixed as follows. One milliliter of 4% paraformaldehyde was added to the culture medium and incubated for 10 minutes at room temperature. The medium and paraformaldehyde was removed, 1 ml of 4% paraformaldehyde was added to each chamber and incubated for 10 minutes at room temperature. The paraformaldehyde was removed again and the chambers were washed twice with PBS 1×. Cells were subsequently stored at 4° C. with 1 ml of PBS 1× in each well.

### Example 10

### Pituitary Adenomas Contain Sphere-Growing Cells that have Self-Renewal Ability

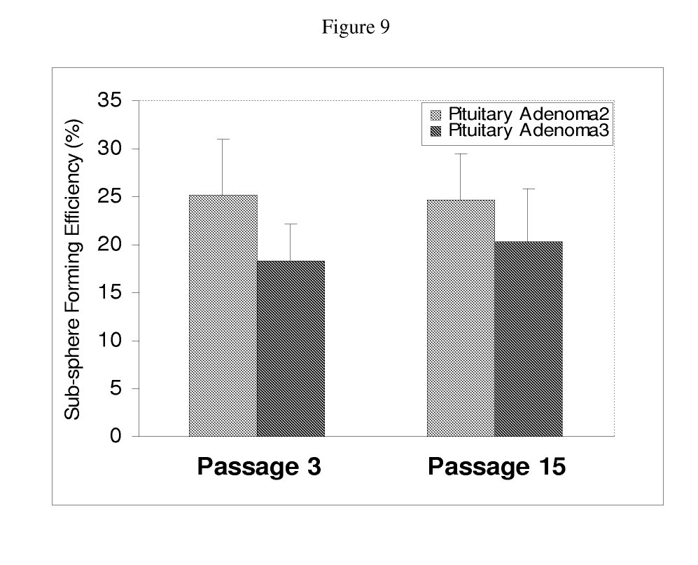

Primary pituitary adenoma cells were cultured in defined neural stem cell medium with growth factors (EGF, 20 ng/ml and bFGF, 20 ng/ml, PeproTech Inc, Rocky Hill, N.J.). Sphere-growing cells were observed in the primary cells after 7-14 days culture. In the cultures (see FIG. 8), some areas were growing as monolayer cells (a & b). Sphere-forming cells were also observed in the cultures (c & d). These spheres were morphologically similar to cancer stem cell spheres in the human glioblastoma cultures. The sphere-growing cells in the culture became free-floating spheres as the culture continued (e & f). The free-floating spheres were passaged in the defined neural stem cell culture media for more than thirty passages without morphological and cell doubling-time changes. The free-floating spheres formed sub-spheres after dissociating into single cells (g & h). These characteristics indicate the self-renewal ability of the sphere-growing pituitary adenoma cells. The images shown on the left panel were from pituitary adenoma No. 2, which is a null-cell macroadenoma (a, c, e & g). The images shown on the right panel were from pituitary adenoma No. 3, which is a somatotroph GH positive adenoma (b, d, f & h).

### Example 11

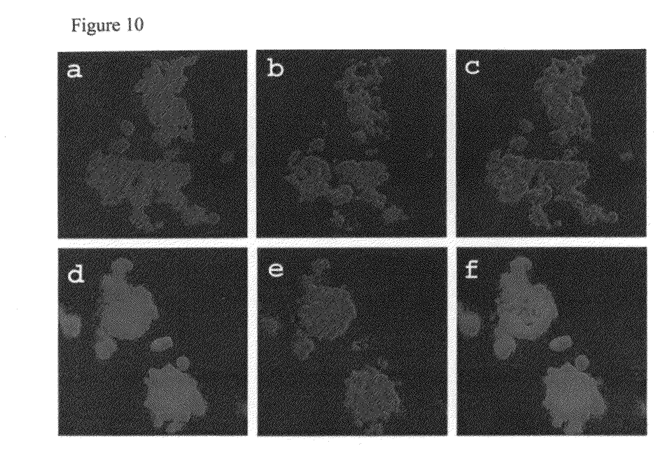

To confirm the self-renewal ability of the adenoma tumor spheres, sub-sphere assay was performed in 96-well culture plates. The spheres were mechanically dissociated into single cells and diluted into culture medium. The suspended cells were seeded into 96-well plates with the dilution that resulted in one cell per well. The wells containing a single cell were identified by checking the culture wells 2 hours post the cell seeding process. Single cells obtained from dissociated spheres could be clonally expanded. After two weeks, culture with medium refreshed every three days, the culture wells were observed and the sub-sphere containing wells were counted. This sub-sphere assay was done with different passages of the sphere cells and experiments were repeated once. (See FIG. 9.)

The ability to expand from a single cell was noted to be consistent at passage 3 and passage 15 in both a somatotroph line as well as a “null cell” line (FIG. 9). Upon differentiation, these cells acquired a large epithelial morphology resembling the pituitary adenoma population. The pituitary adenoma stem cells expressed markers and signaling molecules indicative of stem/progenitor cells and contained cells that generate clonal spheres with comparable expression profiles. The expression of stem associated genes was noted to be down-regulated in a similar fashion between two clones from the same line.

These pituitary spheres expressed members of signaling systems (Notch, Wnt, and Shh) and transcription factors that are essential during pituitary embryogenesis, suggesting that they represent cells with preserved embryonic nature that may function as progenitor cells for new hormone producing cells in the adult gland.

### Example 12

### Sub-Spheres Formed from Single Mother Cell of Tumor Spheres can Express Stem Cell Markers and Hormones

To study whether the self-renewable spheres expressed stem cell genes, the inventor investigated the expression profile of stem cell markers, nestin and CD133. The single sphere cell-derived sub-spheres from pituitary adenomas were stained positive for nestin (1:100, Chemicon, Temecula, Calif.) and CD133 (1:200, Abcam, Cambridge, Mass.). The somatotroph growth hormone (GH) positive adenoma derived tumor spheres were also stained for GH (1:2000, Chemicon, Temecula, Calif.). The primary antibodies were visualized by FITC or Tex-Red conjugated secondary antibodies (Jackson ImmunoResearch, West Grove, Pa.). Cell nuclei were stained by DAPI (Invitrogen, Carlsbad, Calif.).

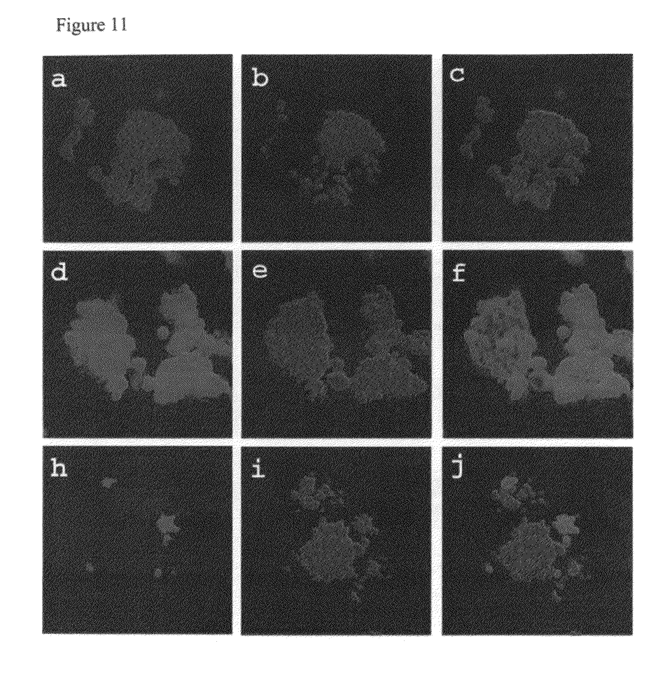

FIG. 10 shows pituitary adenoma No. 2. The sub-spheres expressed stem cell marker genes. Nestin positive spheres were observed as stained in green (a & c). Also, CD133 positive spheres were demonstrated as red (d & f). DAPI were used to localize cell nuclei (b & e). The overlay images are also shown (c & f).

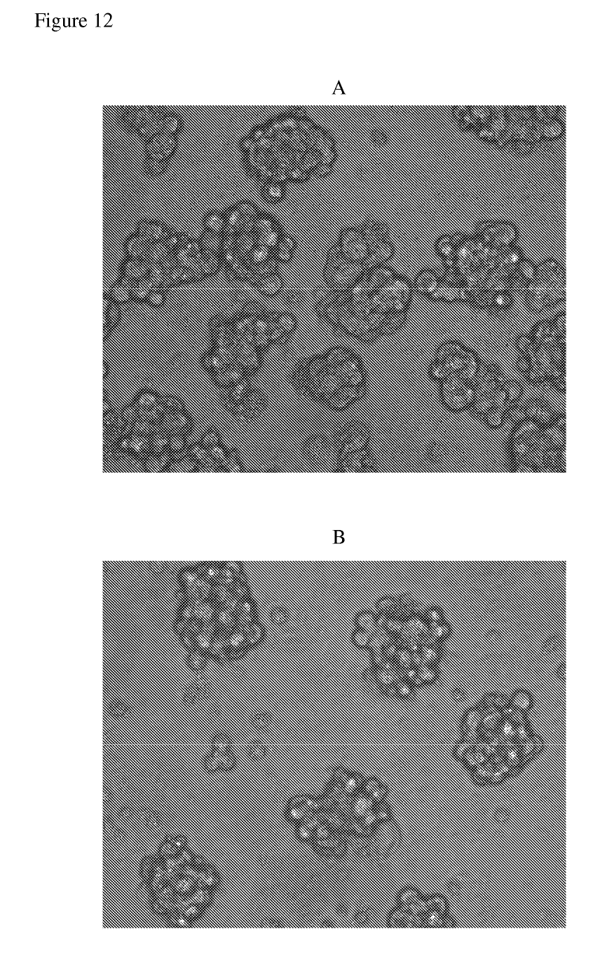

FIG. 11 shows the pituitary adenoma No. 3. The sub-spheres expressed stem cell marker genes. Nestin positive spheres were observed as stained in green (a & c). Also, CD133 positive spheres were demonstrated as red (d & f). Some growth hormone positive cells were identified around the negative stained tumor spheres (h & j). DAPI were used to localize cell nuclei (b, e & i). The overlay images were shown as well (c, f & j).

These data indicate that single sphere cell-derived self-renewable sub-spheres express stem cells markers.

Another confirmation of stem cell-related characteristics that belong to pituitary adenoma stem cells obtained in the cell culture conditions come out from real-time PCR experiments. The inventor investigated by real-time PCR a series of stem cell-related genes, anti-apoptosis proteins and multiple drug resistance genes. It was found stem cells related genes including CD90, Musashi-1 and Notch4 were highly expressed in pituitary adenoma tumor stem cells from a somatotroph GH+ adenoma as compared to the correspondent adherent tumor daughter cells. Similarly anti-apoptotic proteins like BCL-2, cIAP1, NAIP and XIAP are highly expressed in pituitary adenoma spheres as compared to the differentiated adherent daughter cells. There was no significant difference in the expression of multiple drug resistance related genes, including BCRP1, MDR1, MRP1 and MRP3 between tumor stem cells and adherent cells.

The inventor also investigated by real-time PCR also the expression of pituitary genes. Growth hormone, prolactin and Pit-1 showed an increase of 388.02±31.04, 675.59±60.8 and 4.3±0.3 folds, respectively in pituitary adenoma tumor stem-like cells from a somatotroph GH+ adenoma compared to the adherent daughter cells. No detectable mRNA expression of LH, FSH, TSH, ACTH, NeuroD1, Tpit, SF-1 and PROP1 was found in both tumor stem cells and adherent daughter cells. Pituitary tumor-transforming gene (PUG) was expressed in both tumor stem cells and adherent cells. Pit-1 was expressed in both pituitary adenoma spheres and daughter cells of the somatotroph cells. Pit-1 was expressed in the ptiutary adenoma spheres and not in daughter cells from a null cell tumor (PSC 1) or in neither sphere or daughter cells of another null cell tumor (PSC 2). Pit-1 has been found to be a transacting factor for growth hormone and for prolactin. It has been reported the Pit-1 protein expression precedes GH mRNA expression in fetal pituitary gland suggesting that Pit-1 progenitor cells exist in the developing pituitary gland. The results of the expression pattern of somatotroph and null cell Pit-1 in relation to GH expression were consistent with these findings. GATA-2, a zinc finger transcription factor necessary for differentiation and determination of gonadotrophs and thyrotrophs is present only in the adherent cell population and not in the pituitary sphere population.

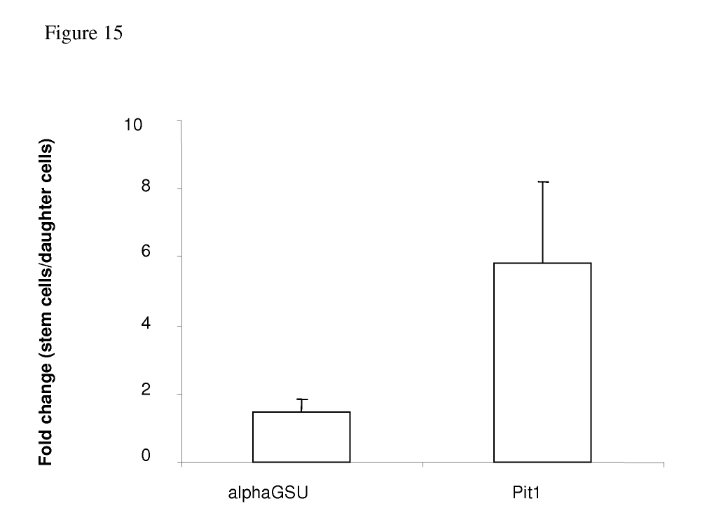

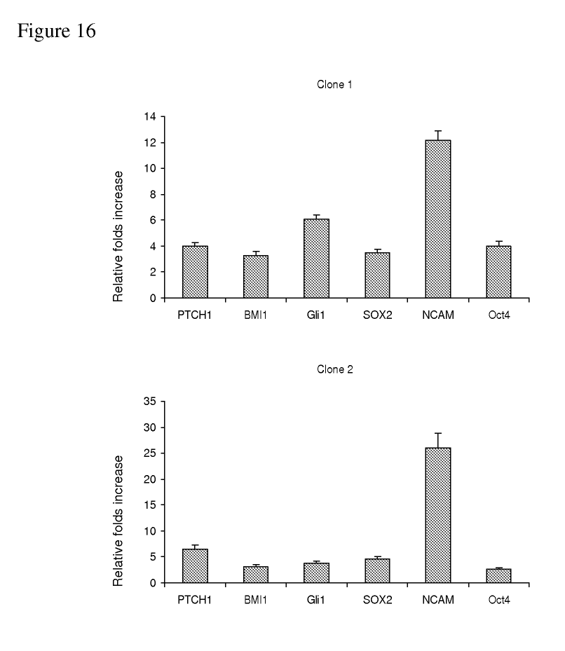

Since cells derived by pituitary tumors, experiments were performed to aim at investigating if cells were able to produce pituitary hormones in vitro. Hormone production by pituitary tumor spheres and differentiated cells was observed. (See FIG. 14.) Relative expression of pituitary-lineage transcription factors in pituitary stem cells compared to that of differentiated pituitary tumor cells was also determined. (See FIG. 15.) ELISA quantification of the hormone production showed a very high production of prolactin and growth hormone in one tumor stem-like cell line derived from a patient with a somatotroph adenoma, which rarely stains for prolactin by immunohistochemistry, while cells derived from the same patient but grown in adherent conditions (presence of serum) were not hormone producing. The levels of the other hormones were also investigated and no expression was detected.

### Example 13

### Single Mother Cell Formed Spheres were Multipotent Upon Differentiation

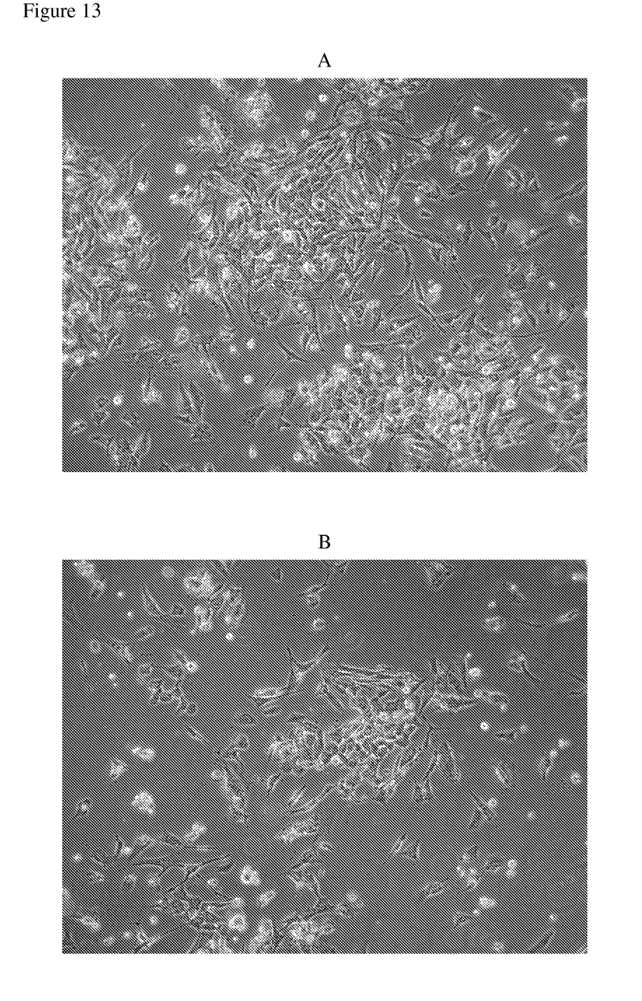

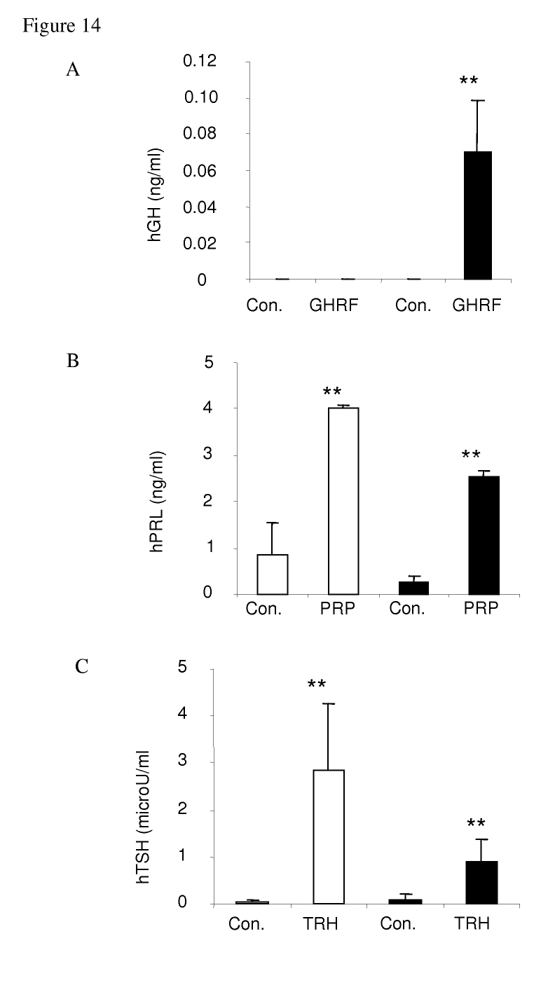

Pituitary tumor stem cells formed spheres resembling neurospheres. Tumor spheres from two pituitary tumors are shown in FIG. 12. Adherent pituitary tumor cells differentiated from the pituitary tumor stem cells. The tumor spheres (stem cells) were switched to differentiation medium and grown for 7-10 days. Two clones are shown in FIG. 13.

### Example 14

### Differentiation of Pituitary Tumor Stem Cells

Pituitary tumor spheres were switched to the differentiation culture medium (DMEM/F12 medium with 2 mM glutamine, 15% horse serum, and 2.5% fetal bovine serum). After the pituitary tumor cells attached to the culture dish, these cells were allowed to grow in the differentiation medium for 7-10 days.

### Example 15

### Stimulated Hormone Production

To determine whether clones derived from the somatotroph tumor and a null cell tumor could be induced to secrete pituitary hormones in response to hypothlalamic releasing factors, pituitary tumor spheres or differentiated pituitary tumor cells were cultured with or without 1×10⁻⁷ M GH-releasing factor (GHRF), 2×10⁻⁷ M PRL-releasing peptide (PRP), and 1×10⁻⁷ M Thyrotropin-Releasing Hormone (TRH) for 24 h. The secreted hormones (GH, PRL, and TSH) in the conditioned media were determined using ELISA kits (Anogen).

### Example 16

### Tumor Spheres Had Different Gene Expression Patterns Compared to their Differentiated Progenies and Monolayer Non-Tumor Cells

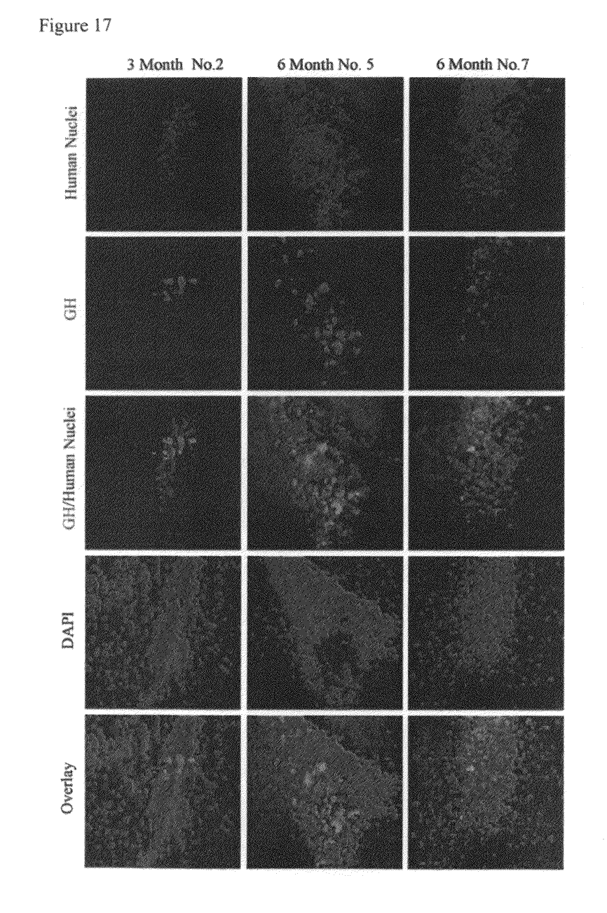

Several stem cell related genes including PTCH1, BMI1, Gli1, SOX2, NCAM and Oct4 were highly expressed on PA No. 3 derived clone 1 tumor stem cells and clone 2 tumor stem cells as compared to those on their differentiated cells. (See FIG. 16.)

### Example 17

### RNA Isolation and cDNA Synthesis

Total RNA was extracted from fresh tumor tissue and isolated CD133 positive and CD133 negative cells using an RNA4PCR kit (Amibion, Austin, Tex.) according to the manufacturer's protocol. For cDNA synthesis, ˜1 μg total RNA was reverse-transcribed into cDNA using Oligo dT primer and iScript cDNA synthesis kit reverse transcriptase. cDNA was stored at ±20° C. for PCR.

### Example 18

### Real-Time Quantitative RT-PCR

Gene expression was quantified by real-time quantitative RT-PCR using QuantiTect SYRB Green dye (Qiagen, Valencia, Calif.). DNA amplification was carried out using Icycler (BIO-RAD, Hercules, Calif.), and the detection was performed by measuring the binding of the fluorescent dye SYBR Green Ito double-stranded DNA. All the primer sets were provided by Qiagen as shown in Table 3. The relative quantities of target gene mRNA against an internal control, GAPDH, was possible by following a ΔC_{T} method. An amplification plot that had been the plot of fluorescence signal vs. cycle number was drawn. The difference (ΔC_{T}) between the mean values in the duplicated samples of target gene and those of GAPDH were calculated by Microsoft Excel and the relative quantified value (RQV) was expressed as 2^{-ΔC}^{_{T}}. The relative expression of each gene presented in each clone was compared between tumor stem cells versus differentiated cells.

TABLE 3
Oligonucleotide primers sequences used for
SYBR Green real-time PCR

| Primer name | | Sequence |
|---|---|---|
| GAPDH | Fw | 5′-CGTCTTCACCACCATGGAGA-3′ |
| | | (SEQ ID NO. 23) |
| | Rev | 5′-CGGCCATCACGCCACAGTTT-3′ |
| | | (SEQ ID NO. 24) |
| GLI1 | Fw | 5′-AGGGAGGAAAGCAGACTGAC-3′ |
| | | (SEQ ID NO. 25) |
| | Rev | 5′-CCAGTCATTTCCACACCACT-3′ |
| | | (SEQ ID NO. 26) |
| PTCH1 | Fw | 5′-TGTGATGTGGGAAAGCAGGAGGAT-3′ |
| | | (SEQ ID NO. 27) |
| | Rev | 5′-ACATGTGCTGGTCTCTGGTTACGA-3′ |
| | | (SEQ ID NO. 28) |
| Oct4 | Fw | 5′-CCTGAAGCAGAAGAGGATCA-3′ |
| | | (SEQ ID NO. 29) |
| | Rev | 5′-CCGCAGCTTACACATGTTCT-3′ |
| | | (SEQ ID NO. 30) |
| NCAM | Fw | 5′-AACCAGCAAGGAAAATCCAA-3′ |
| | | (SEQ ID NO. 31) |
| | Rev | 5′-AGGAGCAGGACGAAGATGAC-3′ |
| | | (SEQ ID NO. 32) |
| SOX2 | Fw | 5′-ACGAGCTCGCAGACCTAGAT-3′ |
| | | (SEQ ID NO. 33) |
| | Rev | 5′-GTGGGAGGAAGAGGTAACCA-3′ |
| | | (SEQ ID NO. 34) |
| BMI1 | Fw | 5′-GGAGACCAGCAAGTATTGTCCTTTTG-3′ |
| | | (SEQ ID NO. 35) |
| | Rev | 5′-CATTGCTGCTGGGCATCGTAAG-3′ |
| | | (SEQ ID NO. 36) |

### Example 19

### Pituitary Tumor Spheres can Form New Tumors Upon Intracranial Implantation

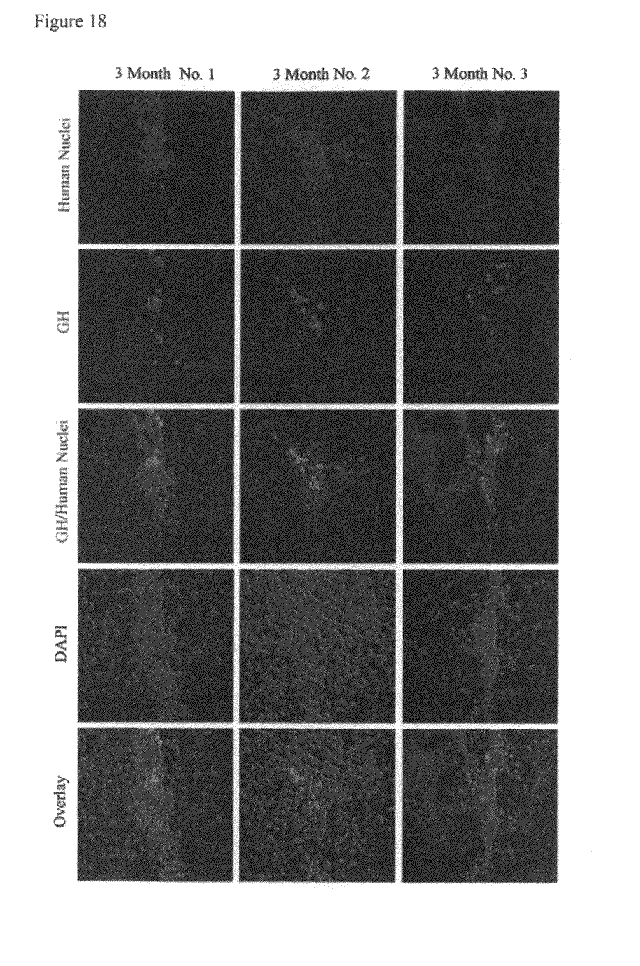

To study whether the pituitary tumor spheres have the ability to form new tumors in in vivo environment, tumor spheres (1×10⁴ cells per mouse) or monolayer non-sphere cells (1×10⁵ cells per mouse) were stereotactically implanted into the right hemisphere of NOD/SCID mice. Three months post the intracranial implantation, human-specific cells were identified in the brains of mice that received tumor spheres implantation. However, there was no human-specific cell found within the brains of mice that received monolayer non-sphere cells implantation. Six months post the intracranial implantation, larger areas of human-specific cell masses were identified in the brains of mice that received tumor spheres implantation compared with that of three months post the intracranial tumor spheres implantation. There were still no human-specific cells found within the brains of mice that received monolayer non-sphere cells. The identification of human-specific cells within the mice brains was performed by immunostaining with human-specific nuclei antibody against human cell nuclei (1:100, Chemicon, Temecula, Calif.). The antibody against human growth hormone (1:2000, Chemicon, Temecula, Calif.) was used to identify GH positive cells within the xenograft tumor mass. (See FIG. 17.)

### Example 20

### Pituitary Tumor-Forming Ability was Confirmed by Serial In Vivo Transplantation

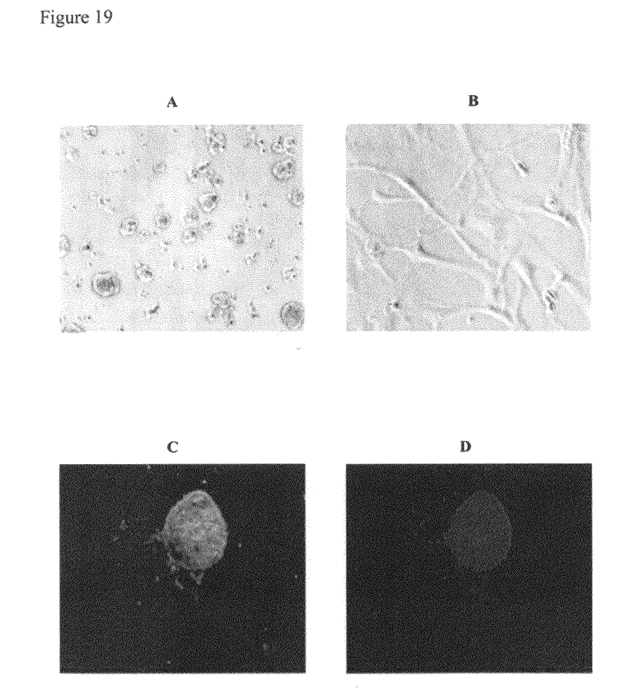

To investigate whether the ability to form new pituitary adenoma tumors was serially transplantable, the sphere-generated tumor masses within the NOD/SCID brains were harvested after 6 months of the intracranial implantation. The harvested tissues were primarily cultured as that of culturing human pituitary adenoma cells described herein. Sphere-growing cells were identifiable in the culture and the spheres can be propagated as free-floating spheres in stem cell culture medium. These spheres cells were re-transplanted into the brains of NOD/SCID mice (1×10⁴ cells per mouse). Three months post the transplantation, the mice were killed and the brain tissues were processed for human-specific cell identification. However there was no human-specific cell found within the brains of mice that received monolayer non-sphere cells implantation. Six month post the intracranial implantation, bigger areas of human-specific cell masses were identified in the brains of mice that received tumor spheres implantation compared with that of three months post the intracranial tumor spheres implantation. There were still no human-specific cells found within the brains of mice that received monolayer non-sphere cells. Some cells within the masses were human growth hormone positive as well. (See FIG. 18.) The identification of human-specific cells within the mice brains was performed by immunostaining with human specific nuclei antibody against human cell nuclei.

In more detail, NOD/SCID mice were implanted either pituitary adenoma No 2 tumor stem cells (1×10⁴/mouse, group 1), pituitary adenoma No 3 tumor stem cells (1×10⁴/mouse, group 2), pituitary adenoma No 2 adherent cells (1×10⁵/mouse, group 3), pituitary adenoma No 3 adherent cells (1×10⁵/mouse, group 4), or human GBM tumor cells U87 (1×10⁴/mouse, group 5) in the right forebrains. One mouse from each of the five groups was euthanized at 6 weeks post-implantation. The euthanized mice forebrains around the implantation site were harvested under a dissection microscope. The dissected samples were homogenized. Human growth hormone specific ELISA kit was used to detect the human GH level of the homogenized tissues.

Three months post-implantation, one animal from each of the above implanted mice (group 1-4) was euthanized and brains were harvested. The brains were subjected for analysis to identify human cells growing within the NOS/SCID mice brains. Immunostaining of the brain sections was performed with human-specific nuclei antibody against human cell nuclei and also with antibody against human specific growth hormone. Human specific cells were found within the mouse brain that was implanted with pituitary adenoma No 3 tumor stem cells. There was no human-specific nuclei immune-positive cell identified within the brains of the mice that were implanted with either pituitary adenoma No 2 tumor stem cells, pituitary adenoma No 2 adherent cells, or pituitary adenoma No 3 adherent cells.

Six months post-implantation, two animals from pituitary adenoma No 3 tumor stem cells implanted mice were euthanized. Another three animals either from pituitary adenoma No 2 tumor stem cells, pituitary adenoma No 2 adherent cells, or pituitary adenoma No 3 adherent cells implanted mice were also euthanized. The brains from the euthanized animals were harvested and subjected for analysis to identify human cells growing within the NOS/SCID mice brains. Immunostaining of the brain sections was performed with human-specific nuclei antibody against human cell nuclei and also with antibody against human specific growth hormone as described previously. Human specific cells were found within the mice brains that were implanted with pituitary adenoma No 3 tumor stem cells. There was no human-specific nuclei immune-positive cell identified within the brains of the mice that were implanted with either pituitary adenoma No 2 tumor stem cells, pituitary adenoma No 2 adherent cells, or pituitary adenoma No 3 adherent cells.

### Example 21

### Pituitary Tumor Stem Cells are More Resistant to Chemotherapy

Pituitary tumor stem cells and adherent daughter cells were also tested for the resistance to chemotherapeutic treatment. Cells were treated with 50 μM carboplatin and 12.5 μM VP16 for 48 h and the toxicity was assessed by WST-1 proliferation assay. The results showed that pituitary tumor stem cells were significantly more resistant than the adherent cells to the chemotherapeutic treatment.

### Example 22

### Cell Culture

Spheres tumor cells (stem cells) were cultured in Dulbecco's Modified Eagle's Medium (DMEM)/F12 Nutrient mixture (1:1, Gibco/BRL) supplemented with B27 1× (Gibco/BRL), penicillin/streptomycin (200 U/ml; Gibco/BRL), fungizone (250 ng/ml, Gibco/BRL), EGF (20 ng/ml; Peprotech) and bFGF (20 ng/ml; Peprotech).

Adherent cells were cultured in DMEM/F12 (1:1), 10% Foetal Bovine Serum (Omega Scientific), penicillin/streptomycin (200 U/ml) and Glutamax 1× (Gibco/BRL).

Dissection Medium: Hank's Balanced Salt Solution 1× (Gibco/BRL), 0.4% Glucose, 1 mM MgCl₂, 30 mg/L Catalase, 6 mg/L Deferoxamine, 25 mg/L N-acetyl-cysteine, 1.25 mg/L superoxide dismutase, 250 ng/ml fungizone (Gibco/BRL), 110 mg/L sodium piruvate, 10 mM Hepes buffer.

Trypsin solution: Trypsin/0.25% EDTA solution 1× (Gibco/BRL)

### Example 23

### Preparation of Tumor Stem Cells from Pituitary Adenomas and Meningiomas

Pituitary tumor samples were collected within half an hour from the surgical resection as approved by the Institutional Review Board at Cedars Sinai Medical Center. Under a biohazard hood tissues were transferred onto a 50 ml centrifuge tube and weighed using a standard laboratory balance. Part of the tissue was cut and transferred onto labeled cryovials and stored at −80° C. for further assay. The remaining specimen was transferred onto a 100 mm cell culture dish and washed three times with 10 ml of PBS 1× to eliminate blood eventually present.

Tissue was then transferred onto a new 100 mm cell culture dish and dissected in a Dissection medium containing catalase, deferoxamine, N-Acetyl cysteine and superoxide dismutase (see Materials for details) using sterile scissors, razors and forceps. Tissue was cut in very small pieces. The dissected tissue was then transferred onto a 50 ml centrifuge tube and centrifuged at 70×g for 1 min and 30 sec in a bench centrifuge. After discarding the supernatant the dissected tissue was resuspended in 10 ml of a Trypsin solution and digested at 37° C. for at least 10 min (The incubation time with trypsin solution strictly depends on specimen size. If sample is up to 1 g the incubation time has to be increased to 20-25 min. The agitation of the sample is critically for homogeneous digestion.) with constant agitation. Trypsin was then neutralized by adding 30 ml of PBS 1× in the tube. The mixture was then centrifuged at 70×g for 1 min and 30 sec in a bench centrifuge. The digested tissue was resuspended in 10 ml of PBS 1× and triturated by passing it in a tissue sieve (0.38 mm, 40 mesh) and recovering the cells in a 100 mm dish. The recovered cells were then passed throughout a 70 μm cell strainer (BD Biosciences) and collected in a 50 ml centrifuge tube. Cells were centrifuged at 168×g for 5 min and counted using a haemocytometer chamber. Cells were plated at the density of 1×10⁵ cells/ml in a medium containing DMEM/F12 (1:1) (Gibco), 10% FBS (Omega Scientific), penicillin/streptomycin (200 U/ml; Gibco) and Glutamax 1× (Gibco). After 24-48 h medium was changed with a medium containing DMEM/F12 (1:1) (Gibco), B27 1× (Gibco), penicillin/streptomycin (200 U/ml; Gibco), fungizone (250 ng/ml), EGF (20 ng/ml) and bFGF (20 ng/ml) (Cells derived from pituitary adenoma growing as spheres were re-fed every two days with fresh medium containing the growth factors.). For each cell line daughter cells growing adherent in 10% FBS containing medium were prepared.

Meningioma stem cells were isolated and propagated by the same method. Isolating and propagating other stem cells from other benign tumors is prepared by the same method.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. An isolated benign tumor stem cell.

2. The isolated benign tumor stem cell of claim 1, wherein the benign tumor stem cell is a pituitary tumor stem cell.

3. The pituitary tumor stem cell of claim 2, wherein the pituitary tumor stem cell is a pituitary adenoma stem cell.

4. The pituitary adenoma stem cell of claim 3, wherein the pituitary adenoma is selected from the group consisting of prolactinoma, somatotrophic adenoma, adrenocorticotropic hormone (“ACTH”)-secreting adenoma, gonadotrophic adenoma, thyrotropic adenoma, null cell adenoma, and combinations thereof.

5. The isolated benign tumor stem cell of claim 1, wherein the benign tumor stem cell is a meningioma stem cell.

6. The isolated benign tumor stem cell of claim 1, obtained by:
providing benign tumor tissue;
washing the benign tumor tissue;
dissecting the benign tumor tissue;
digesting the benign tumor tissue;
triturating the benign tumor tissue to dissociate benign tumor cells;
culturing the benign tumor cells in a medium comprising EGF and bFGF whereby the benign tumor cells growing as spheres are identified as benign tumor stem cells; and
selecting the benign tumor stem cell.

7. A method of obtaining a population of benign tumor cells, comprising:
providing a population of benign tumor stem cells; and
culturing the population of benign tumor stem cells in differentiation culture medium wherein the population of benign tumor stem cells are induced to differentiate into benign tumor cells.

8. The method of claim 7, wherein the differentiation culture medium comprises DMEM/F12, glutamine, horse serum, and fetal bovine serum.

9. The method of claim 7, wherein the benign tumor cells are pituitary adenoma cells or meningioma cells.

9. The method of claim 7, wherein the benign tumor cells are pituitary adenoma cells or meningioma cells.

10. The method of claim 9, wherein the pituitary adenoma cells are selected from the group consisting of prolactinoma cells, somatotropic adenoma cells, adrenocorticotropic hormone (“ACTH”)-secreting adenoma cells, gonadotrophic adenoma cells, thyrotropic adenoma cells, null cell adenoma cells, and combinations thereof.

11. A method of producing a pituitary hormone, comprising:
providing a population of pituitary stem cells and/or pituitary cells obtained by differentiation of pituitary stem cells;
culturing the population in a culture medium; and
isolating the pituitary hormone from the culture medium or the intracellular contents of the pituitary stem cells and/or pituitary cells obtained by differentiation of pituitary stem cells.

12. The method of claim 11, wherein the pituitary hormone is selected from the group consisting of prolactin, growth hormone, adrenocorticotropic hormone, sexual hormone, and combinations thereof.

13. The method of claim 11, wherein the population is a population of cells selected from the group consisting of prolactinoma cells, somatotropic adenoma cells, adrenocorticotropic hormone (“ACTH”)-secreting adenoma cells, gonadotrophic adenoma cells, thyrotropic adenoma cells, null cell adenoma cells, and combinations thereof.

14. A method of identifying a drug to treat a benign tumor disease condition or a benign tumor related disease condition, comprising:
providing a population of benign tumor stem cells and/or benign tumor cells obtained by differentiation of benign tumor stem cells;
culturing the population in a culture medium;
adding a test compound to the culture medium; and
determining the effect of the test compound on the population,
wherein a test compound having a desired effect is identified as a drug capable of treating the benign tumor disease condition or the benign tumor-related disease condition.

15. The method of claim 14, wherein the benign tumor disease condition or the benign tumor-related disease condition is a pituitary disease condition, a pituitary-related disease condition, a meningioma, or a meningeal related disease condition.

16. The method of claim 15, wherein the pituitary disease condition or the pituitary-related disease condition is selected from the group consisting of pituitary adenoma, amenorrhea, galactorrhea, infertility, hypogonadism, gigantism, acromegaly, Cushing's disease, hyperthyroidism and combinations thereof.

17. The method of claim 14, wherein the pituitary stem cells are obtained from a pituitary adenoma.

18. The method of claim 17, wherein the pituitary adenoma is selected from the group consisting of prolactinoma cells, somatotropic adenoma cells, adrenocorticotropic hormone (“ACTH”)-secreting adenoma cells, gonadotrophic adenoma cells, thyrotropic adenoma cells, null cell adenoma cells, and combinations thereof.

19. The method of claim 14, wherein the population of benign tumor stem cells is obtained by:
providing benign tumor tissue;
washing the benign tumor tissue;
dissecting the benign tumor tissue;
digesting the benign tumor tissue;
triturating the benign tumor tissue to dissociate benign tumor cells;
culturing the benign tumor cells in a medium comprising EGF and bFGF whereby the benign tumor cells growing as spheres are identified as benign tumor stem cells; and
selecting the benign tumor stem cell.

20. A kit for producing a pituitary hormone using pituitary stem cells, comprising:
a population of pituitary stem cells and/or pituitary cells obtained by differentiation of pituitary stem cells;
instructions to use the population to produce the pituitary hormone comprising:
instructions to culture the population in a culture medium; and
instructions to isolate the pituitary hormone from the culture medium or the intracellular contents of the population.

21. The kit of claim 20, wherein the pituitary hormone is selected from the group consisting of prolactin, growth hormone, adrenocorticotropic hormone, sexual hormone, and combinations thereof.

22. The kit of claim 20, wherein the population is a population of cells selected from the group consisting of prolactinoma cells, somatotrophic adenoma cells, adrenocorticotropic hormone (“ACTH”)-secreting adenoma cells, gonadotrophic adenoma cells, thyrotropic adenoma cells, null cell adenoma cells and combinations thereof.
